(19) United States
(12) Patent Application Publication
(10) Pub. No.: US 2024/0366715 A1
(43) Pub. Date: Nov. 7, 2024

(54) rhApoE2 COMPOSITIONS AND METHODS OF USING THE SAME TO TREAT METABOLIC DISEASE AND NEURODEGENERATIVE DISEASE

(71) Applicant: University of Kansas, Lawrence, KS (US)

(72) Inventors: Liqin Zhao, Topeka, KS (US); Xin Zhang, Lawrence, KS (US); HeeJung Moon, Lawrence, KS (US); Teruna Siahaan, Lawrence, KS (US)

(21) Appl. No.: 18/653,385

(22) Filed: May 2, 2024

Related U.S. Application Data
(60) Provisional application No. 63/463,822, filed on May 3, 2023.
(60) Provisional application No. 63/598,295, filed on Nov. 13, 2023.

(51) Int. Cl. A61K 38/17; A61K 9/00; A61P 3/06; A61P 25/28

(52) U.S. Cl. CPC A61K 38/1709; A61K 9/0019; A61P 3/06; A61P 25/28

(57) ABSTRACT

The present disclosure provides compositions including rhApoE2 and methods of using the same to improve neuronal glycolysis, synaptic activity, and learning and memory in subjects with or at risk for neurodegenerative diseases, e.g., Alzheimer's Disease, and/or for preventing or treating a metabolic disease such as diabetes, obesity, and cardiovascular disease.

### CROSS-REFERENCE TO RELATED APPLICATIONS

This application claims the benefit of and priority to U.S. Provisional Patent Application No. 63/463,822, filed May 3, 2023, and U.S. Provisional Patent Application No. 63/598,295, filed Nov. 13, 2023, the entire contents of which are incorporated herein by reference.

### STATEMENT OF GOVERNMENT SUPPORT

This invention was made with government support under grant numbers AG061038, and AG071682 awarded by National Institutes of Health. The government has certain rights in the invention.

### TECHNICAL FIELD

The present technology provides compositions and methods of using recombinant human ApoE2 (rhApoE2) protein as a blood-based lipidome modulator in subjects at high-risk for or suffering from metabolic diseases including diabetes, obesity, cardiovascular disease, and neurodegenerative disease such as Alzheimer's disease.

### SEQUENCE LISTING

The instant application contains a Sequence Listing which has been submitted electronically in XML file format and is hereby incorporated by reference in its entirety. Said XML copy, created on Jun. 25, 2024 is named 104434-0316_SL.xml and is 4,709 bytes in size.

### BACKGROUND

The following description of the background of the present technology is provided simply as an aid in understanding the present technology and is not admitted to describe or constitute prior art to the present technology.

Alzheimer's disease (AD) is a neurodegenerative disease and the most common cause of dementia. The cause of AD is poorly understood, and despite decades of active research, the lack of effective AD treatments underscores the importance of alternative strategies that emphasize neuroprotective mechanisms to promote brain resilience against AD. Current AD treatment options are limited and include immunotherapies targeting beta-amyloid, cholinesterase inhibitors, and N-methyl-D-aspartate receptor antagonists. Accordingly, the identification of additional therapies showing higher efficacy is a major unmet clinical need.

### SUMMARY OF THE PRESENT TECHNOLOGY

In one aspect, the present disclosures provide a method for treating or preventing a neurodegenerative disease in a subject in need thereof comprising administering to the subject an effective amount of a plurality of recombinant ApoE2 (rhApoE2) polypeptides, wherein the plurality of rhApoE2 polypeptides comprise O-linked glycosylation and sialylation modifications and have a molecular weight that is greater than 34 kDa. In any or all embodiments of the methods disclosed herein, the plurality of rhApoE2 polypeptides comprises the amino acid sequence of SEQ ID NO: 1.

Additionally or alternatively, in some embodiments of the methods disclosed herein, the neurodegenerative disease is characterized by altered neuronal metabolism (e.g., neuronal glycolysis) compared to that observed in a healthy control subject, and/or the neurodegenerative disease is Alzheimer's Disease, Parkinson's disease, Huntington's disease, or amyotrophic lateral sclerosis. In some embodiments, the subject comprises an ApoE genotype selected from among ApoE ε3/ε3, ApoE ε2/ε4, ApoE ε3/ε4 and ApoE ε4/ε4.

In any of the preceding embodiments of the methods disclosed herein, the plurality of rhApoE2 polypeptides have molecular weights ranging from 34.05 kDa to 37 kDa.

Additionally or alternatively, in some embodiments, the methods of the present technology further comprise administering an effective amount of a blood-brain barrier (BBB) modulator peptide. In some embodiments, the BBB modulator peptide is an E-cadherin-derived peptide, such as ADTC5 (Cyclo(1,7)Ac-CDTPPVC-NH₂) (SEQ ID NO: 2), or HAVN1 (Cyclo(1,6)SHAVSS) (SEQ ID NO: 3). In certain embodiments, the plurality of rhApoE2 polypeptides and/or BBB modulator peptides is administered intravenously or intranasally.

Also disclosed herein are kits comprising rhApoE2, ADTC5 or HAVN1, and instructions for using the same to treat a neurodegenerative disease.

In another aspect, the present disclosure provides a method for preventing or treating a metabolic disease in a subject in need thereof comprising administering to the subject an effective amount of a plurality of recombinant ApoE2 (rhApoE2) polypeptides comprising SEQ ID NO: 1, wherein the plurality of recombinant rhApoE2 polypeptides comprise O-linked glycosylation and sialylation modifications and have a molecular weight that is greater than 34 kDa. The rhApoE2 protein may be administered intravenously. In some embodiments, the metabolic disease is selected from the group consisting of diabetes, obesity, cardiovascular disease, and Alzheimer's disease. Additionally or alternatively, in some embodiments, the subject comprises an ApoE genotype selected from among ApoE ε3/ε3, ApoE ε2/ε4, ApoE ε3/ε4 and ApoE ε4/ε4.

### BRIEF DESCRIPTION OF THE DRAWINGS

FIG. 1 shows metabolic pathways that can cause bioenergetic and biosynthetic disturbances contributing to AD pathogenesis. Glycolysis is a central metabolic pathway in the cytoplasm of cells that breaks down glucose into two pyruvate molecules, accompanied by a net production of two molecules each of ATP and NADH. Hexokinase (HK) is the first key enzyme in glycolysis responsible for phosphorylating glucose to glucose-6-phosphate (G-6-P). The pentose phosphate pathway (PPP) consists of oxidative and non-oxidative phases. In the oxidative step, G-6-P is converted to ribulose-5-phosphate, producing two molecules of NADPH for each molecule of G-6-P. Ribulose-5-phosphate can then enter the non-oxidative phase of the pathway, where various sugar intermediates can be interconverted. Ribose-5-phosphate is a precursor for nucleotide synthesis essential for DNA and RNA production. Fructose-6-phosphate produced from G-6-P serves as the starting point that diverts 2-3% of glucose to the hexosamine biosynthesis pathway (HBP), a branch of the glycolytic pathway. The HBP is responsible for the synthesis of UDP-N-acetylglucosamine (UDP-GlcNAc), which is used as a substrate for protein posttranslational modifications, particularly O-GlcNAcylation. O-GlcNAcylation plays a regulatory role in various cellular processes, including protein folding, cellular metabolism, and signaling.

FIGS. 2A-2C show that physiologically relevant rhApoE2 sialoglycoprotein was produced using the FreeStyle 293 expression system. FIG. 2A shows a Coomassie Blue stained gel demonstrating >95% purity of rhApoE2 derived from 293-F cells. FIG. 2B shows western blots and a graph demonstrating that rhApoE2 derived from 293-F cells possesses posttranslational modifications consistent with the structural features of physiological ApoE2 protein expressed in human brains. Higher molecular weight ApoE is present in human brain tissue and 293-F cell samples, but not E. coli samples. FIG. 2C shows a western blot of a deglycosylation analysis demonstrating that rhApoE2 derived from 293-F cells is highly glycosylated and sialylated exclusively via O-linkages.

FIGS. 3A-3E show that neuronal metabolic activity was altered by uptake of rhApoE2 as evaluated in ApoE4-expressing primary cortical neurons at 14 or 21 days in vitro (DIV). FIG. 3A shows MAP2 labeled ApoE4-expressing primary cortical neurons at 14 or 21 days in vitro (DIV), demonstrating that cultured neurons had neuronal morphology. FIG. 3B shows western blots and a graph demonstrating that pretreatment with rhApoE2 for 1-2 days resulted in significant neuronal uptake of rhApoE2 at 25 μg/ml and 100 μg/ml concentrations. FIG. 3C shows western blots and a graph demonstrating that rhApoE2 treatment upregulated neuronal Akt activity, as evidenced by significantly increased Akt phosphorylation. FIG. 3D shows western blots and a graph demonstrating that rhApoE2 treatment upregulated neuronal expression of the HK2 protein. FIG. 3E shows western blots and a graph demonstrating that rhApoE2 treatment downregulated endogenous levels of ApoE4 protein. *p<0.05, **p<0.01, ***p<0.001, ****p<0.0001. rhE2, rhApoE2; V, vehicle.

FIGS. 4A-4C shows that rhApoE2 treatment promoted neuronal viability and HK2 protein expression and downregulated endogenous ApoE4 expression against neurotoxic insults in ApoE4-expressing primary cortical neurons. FIG. 4A shows that pretreatment with 25 μg/ml rhApoE2 for 2 days significantly increased neuronal viability against H₂O₂-induced neurotoxicity (100 μM H₂O₂ for 30 min), as demonstrated by measures of both neuronal membrane integrity (LDH release) and metabolic activity (Presto Blue fluorescence). Western blots show rhApoE2 treatment upregulated HK2 protein expression, whereas it downregulated endogenous ApoE4 expression in neurons post-exposure to H₂O₂. FIG. 4B shows that pretreatment with 25 μg/ml rhApoE2 for 2 days significantly increased neuronal viability and HK2 expression and downregulated endogenous ApoE4 expression against oligomeric amyloid β (oAβ)-induced neurotoxicity (5 μM oAβ for 2 days). FIG. 4C shows that ApoE4 neurons were challenged by lipopolysaccharide (LPS) activated microglial conditioned medium (LPS-MCM) with or without 25 μg/ml rhApoE2 for 2 days. HK2 protein expression was significantly upregulated whereas endogenous ApoE4 expression was downregulated in neurons exposed to rhApoE2 compared to neurons exposed to vehicle alone. *p<0.05, **p<0.01, ***p<0.001, ****p<0.0001. V, vehicle.

FIGS. 5A-5C show that rhApoE2 was successfully delivered into the brains of hApoE4KI mice when co-administered with ADTC5 via tail vein injection. FIG. 5A shows the synthesis, purification, and confirmation of IRdye800CW-labeled rhApoE2. FIGS. 5B-5C show IRdye800CW-rhApoE2 co-administered with ADTC5 or vehicle alone to hApoE4KI mice via tail vein injection. 40-60 minutes post-injection, mouse brains were perfused and scanned using near-infrared fluorescent imaging. Representative images show IRdye800CW-rhApoE2 deposition in the brain. Serial optical sections were taken from the superior to the inferior, and from the posterior to the anterior. *p<0.05. A, ADTC5; V, vehicle.

FIGS. 6A-6D show enhanced neuronal metabolic activity and synaptic function in rhApoE2 treated middle-aged hApoE4KI mice. FIG. 6A shows 15-18 month old hApoE4KI mice of both sexes were treated with pure, non-conjugated rhApoE2 (0.2 μmol/kg BW) along with ADTC5 (10 μmol/kg BW), rhApoE2 with vehicle alone, or ADTC5 alone, via tail vein injection, once weekly, for 4 weeks. FIG. 6B shows a western blot and graphs demonstrating that brain delivery of rhApoE2 increased cortical HK2 expression and activity. FIG. 6C shows graphs demonstrating that brain delivery of rhApoE2 promoted cortical synaptic vesicle exocytosis in response to neuronal depolarization induced by KCl (60 mM) stimulation. FIG. 6D shows western blots and a graph demonstrating no differences in CD11b expression were observed among groups, indicating the ADTC5-facilitated brain delivery method was safe. *p<0.05, **p<0.01. BW, body weight; HG, high glucose; rhE2, rhApoE2; V, vehicle.

FIGS. 7A-7E show enhanced spatial learning and memory in rhApoE2 treated aged hApoE4KI mice. FIG. 7A shows 20-21-months-old hApoE4KI mice of both sexes were treated with pure, non-conjugated rhApoE2 (0.2 μmol/kg BW) along with ADTC5 (10 μmol/kg BW), rhApoE2 with vehicle alone, or ADTC5 alone, via tail vein injection, once weekly, for 8 weeks. FIG. 7B shows a graph demonstrating that brain delivery of rhApoE2 promoted cortical HK activity. FIG. 7C shows western blots and a graph demonstrating that brain delivery of rhApoE2 downregulated endogenous ApoE4. FIGS. 7D-7E shows experimental design, graphs, and experimental data demonstrating that brain delivery of rhApoE2 promoted the learning and memory function of mice in both Y-maze two-trial and novel object recognition tests. *p<0.05, **p<0.01. M, male mice; BW, body weight.

FIGS. 8A-8E show improved blood lipid profile and reduced inflammatory markers in rhApoE2 treated aged hApoE4KI mice. FIG. 8A and FIG. 8C show that following the two-month treatment of FIG. 7A, mouse serum was collected and subjected to untargeted lipidomic profiling using ultrahigh-performance liquid chromatography coupled with Q-Exactive spectrometry (UPLC-MS). Lipids with VIP>1.5, fold change (FC)>2.0, and p-value<0.05 are considered significant. The heat map generated from hierarchical clustering analyses displays the relative levels of lipids that were significantly altered by rhApoE2. Color intensity correlates with degrees of increase (red) and decrease (green) relative to the mean lipid ratio. G1: rApoE2-treated male mice group; G2: vehicle alone-treated male control group. G3: rApoE2-treated female mice group; G4: vehicle alone-treated female control group. FIG. 8B and FIG. 8D show volcano plots generated from univariate analyses displays FC and p-value of all detected lipids between G1 and G2 (FIG. 8B) or G3 and G4 (FIG. 8D). Red indicates significantly upregulated lipids (X>1; Y>1.30); green indicates significantly downregulated lipids (X<−1; Y>1.30). FIG. 8E shows that splenic expression of IL-1β was downregulated in male mice treated with rhApoE2 compared to mice treated with vehicle alone. **p<0.01. M, male mice.

FIGS. 9A-9C show relative body weight of ApoE2, ApoE3, and ApoE4 knock-in (KI) mice, with ApoE4 KI mice being obese relative to ApoE2 KI mice. FIG. 9A shows ApoE2, ApoE3, and ApoE4 KI body weight at for male and female mice at 9-10 months of age. FIG. 9B shows ApoE2, ApoE3, and ApoE4 KI body weight at for male and female mice at 21-22 months of age. FIG. 9C shows that male ApoE4 KI mice are obese relative to male ApoE2 KI mice, in part due to dysregulated lipid metabolism. A 22.2 month old male ApoE2 KI weighing 30 grams is shown next to a 22.2 month old male ApoE4 KI weighting 67 grams.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. See also, Strachan & Read, Human Molecular Genetics, Second Edition. (John Wiley and Sons, Inc., NY, 1999).

The present disclosure reveals that exogenous expression of ApoE2 in ApoE4 cells induced a significant increase in hexokinase (HK) and glycolytic function. Increased neuronal HK activity and glycolytic flux are believed to promote neuronal bioenergetics, neuronal viability, and neuronal synaptic strength against neurodegenerative insults, e.g., beta-amyloid toxicity.¹³⁹ Recombinant human ApoE2 (rhApoE2) protein effectively protected primary neurons against H₂O₂ insult, potentially via the upregulation of HK. As disclosed herein, rhApoE2 protein was delivered using ADTC5 peptide as a blood-brain barrier modulator into the brains of human ApoE4 knock-in (hApoE4KI) mice via tail vein injection. rhApoE2 protein was then administrated once every week for 4 weeks in mid-aged hApoE4KI mice, or for 8 weeks in aged hApoE4KI mice. HK was significantly enhanced in the brains treated with rhApoE2 along with ADTC5 in both studies, with no sign of neurotoxicity. In addition, synaptosomal activity was significantly increased in rhApoE2-treated mid-aged hApoE4KI mice. 2-month rhApoE2 treatment promoted synaptosomal glycolytic function and spatial recognition memory in aged hApoE4KI mice. Moreover, rhApoE2 treatment reduced IL-1β expression in aged mice spleen, suggesting its potential efficacy in ameliorating inflammatory process associated with ApoE4. Collectively, these data provide a novel approach to bolster brain resilience in the prevention and early intervention of AD.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, “gene” refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full-length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, “sample” or “biological sample” refers to a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leukocytes, and platelets), serum and plasma.

As used herein, “subject”, “patient”, or “individual” refers to an individual organism, a vertebrate, a mammal, or a human. In some embodiments, the subject, patient or individual is a human.

As used herein, “therapeutic agent” refers to a compound that, when present in an effective amount, produces a desired therapeutic effect on a subject in need thereof.

As used herein, “treating” or “treatment” refers to the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Neuronal Metabolic Dysfunction Precedes AD Onset

An adult brain makes up only 2% of the body's mass, yet it uses about 20-25% of the oxygen and glucose consumed at rest.¹ The brain's significant energy-consuming quality is primarily attributed to the highly active and complex processes involved in neural transmission. Failure to maintain basal energy levels, such as during hypoglycemia, can potentially induce synaptic loss and cognitive impairment within a few minutes, thus rendering the brain exceedingly vulnerable to energy deficit-mediated damage.²⁻⁴ Accumulated evidence indicates that, in the development of Alzheimer's disease (AD), pathophysiological changes can occur up to 20-30 years before clinical symptoms manifest. Metabolic dysfunction has been recognized as a prominent anomaly in the brain during this prodromal stage.⁵⁻⁹ The cerebral metabolic rate of glucose (CMRglc) is a critical indicator of neuronal and synaptic activity.¹⁰⁻¹³ Using positron emission tomography (PET) imaging and the use of 2-[18F] fluoro-2sdeoxy-D-glucose (FDG) as the tracer, studies have shown that nearly all clinical AD symptoms are accompanied by a significant reduction of CMRglc, and the extent and topography correlate closely with symptom severity.¹⁴ Among individuals with mild cognitive impairment (MCI), significantly decreased glucose metabolism has also been observed in AD-vulnerable brain regions such as the hippocampus, posterior cingulate cortex, and temporal cortex.^{7,15} This hypometabolic condition predicts progression from MCI to AD with greater than 80% accuracy. Additionally, compared to noncarriers, individuals carrying an ApoE4 allele without dementia exhibited a mild but definite reduction in CMRglc similar to the typical AD pattern.^{6, 15-18} Considerable research has sought to understand the biological basis of the impaired glucose metabolism observed in the brains of AD patients and high-risk individuals.

### Neuronal Glycolysis

The human brain depends mostly on glucose as its fuel source, putting it at significant risk for neuronal dysfunction when glucose is in short supply. Apart from glucose, the brain can also utilize other energy substrates. For example, ketone bodies can provide energy,^{19,20} but their fuel role is considered minor, except in cases of starvation and glucose deprivation. Studies indicate that during heightened neuronal activity, neuronal glycolysis increases to meet the energy demand and supply intermediates for synthesizing neurotransmitters.²¹⁻²³ Glycolysis is a central metabolic pathway that occurs in the cytoplasm of cells and is responsible for breaking down glucose into two pyruvate molecules, along with a net production of two molecules each of ATP and NADH. Mitochondrial respiration then metabolizes the pyruvates. Glycolysis has been well-characterized for its crucial roles in supporting both bioenergetics and biosynthesis in the brain. Even though cells mainly rely on oxidative phosphorylation as their primary source of energy, the rate of ATP generated in the glycolytic pathway has been observed at a much higher level than oxidative phosphorylation in several different types of cells, including neurons, astrocytes, and microglia.²⁴⁻²⁹ The high speed of ATP production by glycolysis is essential for neural processes when rapid ATP production is needed, such as during action potentials and synaptic transmission that involve quick and transient changes in electrical and chemical activity occurring in neurons. Producing energy is not the sole purpose of glycolysis. Various metabolic intermediates generated in the glycolytic pathway flow into different biosynthetic processes, including gluconeogenesis, the pentose phosphate pathway (PPP), the TCA cycle, and lipid metabolism. Thus, the significance of glycolysis extends beyond rapid energy generation to facilitate nutrient assembly into essential precursors and promote cellular homeostasis.

### Neuronal Glycolytic Deficits in AD Patients

Glycolytic deficits have long been observed as a prominent metabolic dysfunction in early AD, evidenced by a much greater decline in cerebral glucose utilization compared to the decrease in cerebral blood flow and the cerebral metabolic rate of oxygen consumption.^{30, 31} These earlier observations have been solidified by recent findings demonstrating that reduced glycolytic flux correlates closely with the severity of the disease, with more plaques and tangles found in the brains of AD patients.^{32,33} A recent study of 42 individuals aged 53-88 years with either preclinical or symptomatic stages of AD revealed a close relationship among brain glycolysis, amyloid burden, and tau deposition. The data showed that reduced synaptic plasticity was related to the loss of brain glycolysis, which may have promoted tauopathy in individuals with amyloid burden.³³ Moreover, studies have shown that many glycolytic elements, including glycolytic enzymes, glycolytic intermediates, and amino acids produced in the glycolytic pathway, are altered in AD. Using the autopsy cohort of the Baltimore Longitudinal Study of Aging, the Thambisetty group investigated whether AD pathogenesis was associated with abnormalities in brain glucose homeostasis. Glucose concentration and the ratios of glycolytic amino acids (serine, glycine, and alanine) to glucose were measured as indicators of cerebral glycolytic activity. The results showed that elevated brain glucose levels and reduced glycolytic flux were related to the severity of AD pathology and the expression of AD symptoms. These findings led the authors to conclude that impaired glycolysis may be intrinsic to glucose metabolic dysfunction inherent in AD pathogenesis.³² Another study that analyzed 122 metabolites in the CSF of AD and non-AD subjects found that only intermediates of glycolysis, such as dihydroxyacetone phosphate (DHAP) and phosphoenolpyruvate (PEP), were significantly decreased in AD patients. The reduction of these glycolytic intermediates also exhibited a positive correlation with brain levels of Aβ₁₋₄₂ and Aβ₁₋₄₂/Aβ_{1-40.}³⁴ Preclinical studies have provided strong evidence for the role of glycolysis in the pathogenesis of AD as well. For example, studies using h-tau mice, which expressed all human tau isoforms, found that reduced glucose utilization, possibly via the downregulation of glycolysis, directly triggered tauopathy, leading to synaptic dysfunction and behavior deficits.³⁵

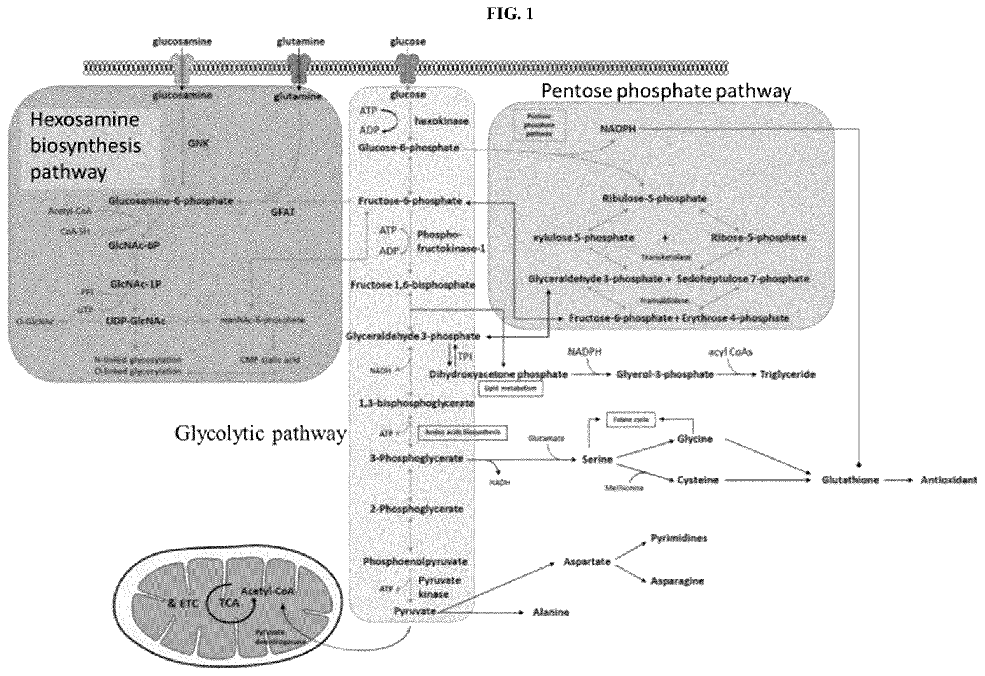

Another area associated with glycolysis that can contribute to AD pathogenesis is the hexosamine biosynthesis pathway (HBP), a side branch of glycolysis. Notably, O-linked N-acetylglucosamine (O-GlcNAc) is significantly enriched in the brain, particularly at the synapses, suggesting a critical role of the HBP in synaptic transmission.³⁶⁻³⁹ Decreased O-GlcNAcylation has been inversely correlated with increased amyloidogenic APP metabolism and tau phosphorylation.⁴⁰⁻⁴⁴ Furthermore, glycolytic deficits can potentially cause decreased NADPH, an essential product of the PPP. As a vital reducing agent, NADPH is utilized in various biosynthetic processes (e.g., fatty acid and cholesterol synthesis) and cellular defense against oxidative stress. NADPH is particularly critical in glutathione metabolism, as indicated by the GSS/GSSG ratio (GSH:reduced glutathione; GSSG:oxidized glutathione), a significant mechanism maintaining cellular redox homeostasis. Therefore, NAPDH deficiency reduces the antioxidant power of the cellular glutathione redox cycle and leads to oxidative damage, another common feature of AD. In summary, and without wishing to be bound by theory, it is believed that glycolytic deficits, a potentially major cause of decreased glucose metabolism in AD brains, can cause both bioenergetic and biosynthetic disturbances that disrupt metabolic and synaptic homeostasis, leading to abnormal protein deposition and cognitive decline (FIG. 1).

### Apolipoprotein E (ApoE)

Endogenous human ApoE is a polypeptide comprising 317 amino acids (aa). The first 18 AAs comprise a cleavable signal peptide, which is absent in rhApoE2. Cleaved ApoE (299 AAs) has a molecular weight of 34 kDa and exists in 3 major isoforms, ApoE2, ApoE3 and ApoE4. These isoforms differ from one another at aa positions 112 and 158. While ApoE2 contains two cysteine residues, ApoE3 contains a cysteine and an arginine, and ApoE4 contains two arginine residues at both sites, respectively. ApoE4 presents AD risk in a gene dose-dependent manner, with ε3/ε4 heterozygotes having AD risk increased by three times and ≥4/ε4 homozygotes having up to 15 times more chance of developing AD. ApoE3 is the most common isoform, associated with a neutral risk. ApoE2 is under-represented in the population but is thought to be associated with a lower propensity for AD.

O-Glycosylation and sialylation of ApoE2 can occur on residues Thr-8, Thr-18, Thr-194, Thr-289, Ser-290, and Ser-296, adding approximately 200-500 Da molecular weight per glycosylation. ApoE2 glycoforms have molecular weights ranging from about 34 kDa to about 37 kDa.

ApoE expression in the brain is heterogeneous. Although astrocytes are known to be the primary source of ApoE production, followed by microglia,^{45,46} there is substantial evidence showing that ApoE is also expressed in neurons, although at lower levels than glial cells. It is estimated that the neuronal source of ApoE represents approximately 20% of total ApoE in the cortex^{47,48} ApoE protein and mRNA have been observed in hippocampal and cortical neurons in human brains and human ApoE gene-targeted replacement mice brains.^{49,50} Moreover, studies suggest that human neurons can synthesize ApoE in the presence of astrocytes, and neuronal production of ApoE is likely regulated by a feedback mechanism controlled by the neuron itself.⁵¹ Although the exact role of ApoE in neurons is not fully understood, the markedly upregulated expression of ApoE observed in injured or stressed neurons suggests a potentially important role of neuronal ApoE in neuronal repair and neuroprotection mechanisms.⁵²⁻⁵⁴ Recent studies suggest that ApoE isoforms differentially modulate brain bioenergetics. Of particular significance, several lines of in vitro and in vivo evidence suggests that ApoE plays a significant role in the regulation of neuronal hexokinase expression and glycolytic activity, with ApoE2 upregulating and ApoE4 downregulating compared to ApoE3, and the ApoE isoform-mediated varying glycolytic profiles appear to have a strong influence on neuronal health and aging.⁵⁵⁻⁵⁹ Additionally, neurons with a higher level of glycolysis are more resistant to beta-amyloid toxicity, suggesting a neuroprotective effect mediated by neuronal glycolysis.^{60,61}

### Ceramides and Triglycerides in Metabolic Syndrome and AD

Ceramides in metabolic syndrome and AD). Ceramides are bioactive lipids involved in the formation of ceramide-rich lipid rafts within the cell membrane and in the regulation of cellular processes related to apoptosis, cell proliferation, and differentiation. Ceramides have been widely indicated for their profoundly lipotoxic roles in the pathogenesis of a variety of metabolic diseases, cancer, and neurodegeneration.^{62,63} A ceramide-centric view posits that ceramide accumulation drives the onset of insulin resistance, underlying obesity-associated risks for the development of type 2 diabetes and cardiovascular disease.^{64,65} Elevated levels of ceramides in the circulation have been shown to independently and comprehensively predict major adverse cardiovascular events in patients with and without coronary artery disease, and the ceramide risk score outperforms LDL cholesterol.⁶⁶⁻⁶⁸

For these reasons, the Mayo Clinic has recently started offering the MI-HEART blood test that quantifies plasma ceramides as a novel biomarker of cardiovascular risk prediction (https://news.mayocliniclabs.com/ceramides-miheart/). High blood ceramide levels have also been shown to predict cognitive decline and increased risk of Alzheimer's disease (AD).69-71 Of particular note, in a more recent study (626 men and 366 women, aged 55 years older, with an mean follow-up of 13-15 years), it was found that the plasma ceramide-AD association differed by sex and ApoE genotype, and higher ceramide levels were associated with an increased risk of AD among men, but not women.⁷² Furthermore, studies conducted in rodents revealed that inhibition of ceramide biosynthesis or increasing ceramide degradation by genetic manipulations substantially reduced insulin resistance, inflammation, mitochondrial stress, and improved systemic metabolism.⁷³⁻⁷⁶ Taken together, the existing literature highlights the enormous promise of ceramide-reduction therapies, in particular, blood-based, as a systemic intervention for preventing and treating not only a broad spectrum of peripheral metabolic disorders but also neurodegenerative diseases such as AD.

Triglycerides in metabolic syndrome and AD. Triglycerides (TGs) play an important role in storing and transporting fatty acids (FAs) and serve as a major source of energy in the human body. FAs are commonly classified into three groups based on the number of double bonds contained in the long hydrocarbon chain: saturated FAs (SFAs) containing zero double bonds, monounsaturated FAs (MUFAs) containing one double bond, and polyunsaturated FAs (PUFAs) containing two or more double bonds. Although the underlying mechanisms have yet to be fully defined, it is well documented that the metabolism and behavior of the various types of FAs differ greatly.

SFAs are generally considered to be lipotoxic and are associated with an increased risk for metabolic diseases such as diabetes and heart disease.^{77,78}. With respect to the health impact of MUFAs, the current literature presents a mixed picture. Even though there is substantial evidence in support of their health benefits and their roles in counteracting SFAs-induced cytotoxicity,⁷⁹⁻⁸¹ negative effects have also been associated with MUFAs. For example, oleic acid was shown to induce lipotoxicity in beta cells⁸²⁻⁸⁴ and mediate α-synuclein cytotoxicity in Parkinson's disease models.^{85,86} In contrast, long chain PUFAs, such as eicosapentaenoic acid (EPA) and docosahexaenoic acid (DHA), are widely regarded to be lipoprotective and promote healthy aging throughout life.^{87,88} The brain is particularly enriched with EPA and DHA, which are found in abundance in neuronal membranes and play vital roles in neuronal maturation, neurite outgrowth, and neurotransmitter release.⁸⁹⁻⁹³

EPA and DHA have also been broadly indicated for their beneficial effects in the aging brain, including maintaining white matter microstructural integrity and strengthening the neural circuits that are vulnerable to age-related decline.⁹⁴⁻⁹⁸ In older adults, lower levels of DHA in the blood have been associated with smaller brain volumes, a marker of accelerated brain aging.⁹⁹ Consistent with the neuroprotective properties of PUF As in the brain, a recent study of 689 participants from the Alzheimer's Disease Neuroimaging Initiative cohort revealed a significant association between long-chain-PUFAs-containing TGs (PUTGs) and mild cognitive impairment (MCI) and AD. Lower serum levels of PUTGs were observed and associated with increased brain atrophy and decreased CSF amyloid-beta concentrations in individuals with MCI and AD compared to cognitively normal controls, and these associations were more pronounced in ApoE4 carriers.¹⁰⁰

Traditionally, in a health-care setting, the amount of total TG in the blood is usually measured in a TG test and used as a biomarker for certain disease risk prediction. In fact, hypertriglyceridemia, a condition characterized by high TGs in the blood, can contribute to arteriosclerosis, increasing the risk of stroke and heart attack as well as inflammation of the pancreas. However, under certain circumstances, elevated levels of TGs in the blood may signal a positive metabolic response of the human body. For example, TG accumulation has been shown to mediate the rescuing effect of unsaturated FAs against SFAs-induced apoptosis.⁷⁹ Taken together, these studies highlight the importance of more detailed analyses of the composition of TGs beyond the concentration of TGs-such as the types of FAs and associated saturation status and chain length—for improved accuracies in the assessment of disease risks and therapeutic efficacies. It is conceivable that increased levels of SFAs-containing TGs may very likely have a different health impact than PUFAs-containing TGs. Moreover, as a component of chylomicrons and very low-density lipoproteins, ApoE is responsible for the transport of TGs in the blood. ApoE alleles have been shown to differentially influence TG metabolism; however, the results have been largely inconsistent, which could be in part due to different types of TGs involved in different conditions.¹⁰¹⁻¹⁰⁵ Furthermore, like many other lipid species, including ceramides, sex plays a role in TG metabolism as well. A sex-specific TG signature has been associated with obesity, with males exhibiting higher serum TG levels and more insulin resistance compared to females.¹⁰⁶ Thus, overall, a more comprehensive analytical approach, taking into account of both the composition and concentration of TGs and ApoE genetic status as well as sex, is expected to yield a better understanding of the roles of TGs in human health and disease including AD.

### Formulations of the Present Technology

According to the methods of the present technology, the rhApoE2 polypeptides and/or BBB modulator peptides the present technology can be incorporated into pharmaceutical compositions suitable for administration. The pharmaceutical compositions generally comprise recombinant or substantially purified peptides and a pharmaceutically-acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the rhApoE2 polypeptides and/or BBB modulator peptides, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. The rhApoE2 polypeptides and/or BBB modulator peptides named in this technology can be present in unsalified or unesterified form, or in salified and/or esterified form, and the naming of such rhApoE2 polypeptides and/or BBB modulator peptides is intended to include both the original (unsalified and unesterified) compound and its pharmaceutically-acceptable salts and esters. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the rhApoE2 polypeptides and/or BBB modulator peptides disclosed herein, use thereof in the compositions is contemplated. Supplementary active compounds can also be incorporated into the compositions.

A pharmaceutical composition of the present technology is formulated to be compatible with its intended route of administration. The compositions including the rhApoE2 polypeptides and/or BBB modulator peptides of the present technology can be administered by parenteral, topical, intravenous, oral, intratumoral, subcutaneous, intraarterial, intradermal, transdermal, rectal, intracranial, intrathecal, intraperitoneal, intranasal; or intramuscular routes, or as inhalants. The rhApoE2 polypeptides and/or BBB modulator peptides can optionally be administered in combination with other agents that are at least partly effective in treating various neurodegenerative disease pathologies.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating the rhApoE2 polypeptides and/or BBB modulator peptides of the present technology in the required amount in an appropriate solvent with one or a combination of ingredients enumerated above, as required, followed by filtered sterilization. Generally, dispersions are prepared by incorporating the rhApoE2 polypeptides and/or BBB modulator peptides into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The peptides of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the rhApoE2 polypeptides and/or BBB modulator peptides described herein can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the rhApoE2 polypeptides and/or BBB modulator peptides are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the rhApoE2 polypeptides and/or BBB modulator peptides is formulated into ointments, salves, gels, or creams as generally known in the art.

The rhApoE2 polypeptides and/or BBB modulator peptides can also be prepared as pharmaceutical compositions in the form of suppositories (e.g., with conventional suppository bases such as cocoa butter and other glycerides) or retention enemas for rectal delivery.

In one embodiment, the rhApoE2 polypeptides and/or BBB modulator peptides is prepared with carriers that will protect the rhApoE2 polypeptides and/or BBB modulator peptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

Sterile injectable solutions can be prepared by incorporating the compositions of the presently disclosed subject matter, e.g., a composition comprising rhApoE2 polypeptides and/or BBB modulator peptides, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the rhApoE2 polypeptides and/or BBB modulator peptides of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The skilled artisan can readily determine the amount of rhApoE2 polypeptides and/or BBB modulator peptides and optional additives, vehicles, and/or carrier in compositions and to be administered in methods of the presently disclosed subject matter. Typically, any additives (in addition to the rhApoE2 polypeptides and/or BBB modulator peptides and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Methods of Treatment of the Present Technology

The following discussion is presented by way of example only, and is not intended to be limiting.

In one aspect, the present disclosure provides a method for treating or preventing a neurodegenerative disease in a subject in need thereof. In some embodiments, the neurodegenerative disease is Alzheimer's Disease, Parkinson's disease, Huntington's disease, amyotrophic lateral sclerosis, or other neurodegenerative disease. In other embodiments, the subject in need displays signs of early, middle, or late dementia. In some embodiments, the neurodegenerative disease is characterized by altered neuronal metabolism (e.g., neuronal glycolysis) compared to that observed in a healthy control subject. In other embodiments the subject comprises an ApoE genotype selected from among ApoE ε3/ε3, ApoE ε2/ε4, ApoE ε3/ε4 and ApoE ε4/ε4. In some embodiments the neurodegenerative disease is AD.

Subjects suffering from a neurodegenerative disease, dementia, or AD can be identified by any or a combination of diagnostic or prognostic assays known in the art. For example, typical symptoms of AD include, but are not limited to, memory problems, thinking and reasoning difficulties, language problems, changes in mood, changes to how things are seen or heard, agitation, pacing, delusions, changes in sleep patterns, and sundowning.

In another aspect, the present disclosure provides a method for treating or preventing a neurodegenerative disease in a subject in need thereof comprising administering to the subject an effective amount of a plurality of recombinant ApoE2 (rhApoE2) polypeptides comprising an amino acid sequence that is at least 80%, at least 85%, at least 90%, at least 95%, at least 99% or 100% identical to SEQ ID NO: 1. In some embodiments, the present disclosure provides a method for treating or preventing a neurodegenerative disease in a subject in need thereof comprising administering to the subject an effective amount of a plurality of rhApoE2 polypeptides comprising SEQ ID NO: 1. In other embodiments, the present disclosure provides a method for treating or preventing a neurodegenerative disease in a subject in need thereof comprising administering to the subject an effective amount of a plurality of rhApoE2 polypeptides consisting of SEQ ID NO: 1. In some embodiments, the rhApoE2 polypeptide comprises O-linked glycosylation and sialylation modifications and has a molecular weight that is greater than 34 kDa.

In one aspect, the present disclosure provides a method for preventing or treating a metabolic disease in a subject in need thereof comprising administering to the subject an effective amount of a plurality of recombinant ApoE2 (rhApoE2) polypeptides comprising SEQ ID NO: 1, wherein the plurality of recombinant rhApoE2 polypeptides comprise O-linked glycosylation and sialylation modifications and have a molecular weight that is greater than 34 kDa. The rhApoE2 protein may be administered intravenously. In some embodiments, the metabolic disease is selected from the group consisting of diabetes, obesity, cardiovascular disease, and Alzheimer's disease. Additionally or alternatively, in some embodiments, the subject comprises an ApoE genotype selected from among ApoE ε3/ε3, ApoE ε2/ε4, ApoE ε3/ε4 and ApoE ε4/ε4.

In another aspect, the present disclosure provides a method for treating or preventing a metabolic disease in a subject in need thereof comprising administering to the subject an effective amount of a plurality of recombinant ApoE2 (rhApoE2) polypeptides comprising an amino acid sequence that is at least 80%, at least 85%, at least 90%, at least 95%, at least 99% or 100% identical to SEQ ID NO: 1. In some embodiments, the present disclosure provides a method for treating or preventing a metabolic disease in a subject in need thereof comprising administering to the subject an effective amount of a plurality of rhApoE2 polypeptides comprising SEQ ID NO: 1. In other embodiments, the present disclosure provides a method for treating or preventing a metabolic disease in a subject in need thereof comprising administering to the subject an effective amount of a plurality of rhApoE2 polypeptides consisting of SEQ ID NO: 1. In some embodiments, the rhApoE2 polypeptide comprises O-linked glycosylation and sialylation modifications and has a molecular weight that is greater than 34 kDa.

In any of the preceding embodiments, the plurality of recombinant rhApoE2 polypeptides have molecular weights that range from 34.05 kDa to 37 kDa. In some embodiments the plurality of rhApoE2 polypeptides have molecular weights that range from about 34.05 kDa, about 34.1, about 34.2, about 34.3, about 34.4, about 34.5, about 34.6, about 34.7, about 34.8, about 34.9, about 35, about 35.1, about 35.2, about 35.3, about 35.4, about 35.5, about 35.6, about 35.7, about 35.8, about 35.9, about 36, about 36.1, about 36.2, about 36.3, about 36.4, about 36.5, about 36.6, about 36.7, about 36.8, about 36.9 kDa or about 37 kDa. In some embodiments the plurality of rhApoE2 polypeptides comprise a mixture of 1, 2, 3, 4, 5, 6, 7, 8, 9, or more glycoforms, wherein each glycoform has a molecular weight that ranges from about 34.05 kDa, about 34.1, about 34.2, about 34.3, about 34.4, about 34.5, about 34.6, about 34.7, about 34.8, about 34.9, about 35, about 35.1, about 35.2, about 35.3, about 35.4, about 35.5, about 35.6, about 35.7, about 35.8, about 35.9, about 36, about 36.1, about 36.2, about 36.3, about 36.4, about 36.5, about 36.6, about 36.7, about 36.8, about 36.9 kDa or about 37 kDa.

Additionally or alternatively, in some embodiments, the methods of the present technology further comprise administering an effective amount of a blood-brain barrier (BBB) modulator peptide. In some embodiments, the BBB modulator peptide is an E-cadherin-derived peptide. In other embodiments, the E-cadherin-derived peptide is ADTC5 (SEQ ID NO: 2) or HAVN1 (SEQ ID NO: 3).

In certain embodiments, the plurality of recombinant ApoE2 (rhApoE2) polypeptides and/or BBB modulator peptides is administered intravenously or intranasally. Intravenous or intranasal administration can be performed by any means known in the art. In some embodiments, rhApoE2 is administered at 0.2 μmol/kg body weight. In other embodiments, rhApoE2 is administered at between 0.01 and 2 μmol/kg body weight. In some embodiments, the BBB modulator peptide is co-administered at 10 μmol/kg body weight. In other embodiments, the BBB modulator peptide is co-administered at between 0.5 and 100 μmol/kg body weight.

The therapeutically effective amounts or suitable dosages of rhApoE2 polypeptide and/or BBB modulator peptide depends upon a number of factors, including the nature of the severity of the condition to be treated, the route of administration, and the age, weight, general health, and response of the individual patient. In some embodiments, the therapeutically effective amount may be determined during pre-clinical trials and clinical trials by methods familiar to physicians and clinicians. In other embodiments, the suitable dose level is one that achieves a therapeutic response as measured by the Global Deterioration Scale and/or other clinical measures of disease progression. In some embodiments, therapeutic response is measured quantitatively or qualitatively by one or more techniques selected from the group comprising electroencephalogram (EEG), neuroimaging, functional MRI, structural MRI, diffusion tensor imaging (DTI), [18F] fluorodeoxyglucose (FDG) PET, agents that label amyloid, [18F] F-dopa PET, radiotracer imaging, volumetric analysis of regional tissue loss, multimodal imaging, and biomarker analysis. In other embodiments, the suitable dose level is one that achieves therapeutic response and also minimizes any side effects associated with the administration of the therapeutic agent.

Suitable daily dosages of rhApoE2 polypeptide and/or BBB modulator peptide can generally range, in single or divided or multiple doses, from about 10% to about 120% of the maximum tolerated dose of each agent. In some embodiments, the suitable dosages of rhApoE2 polypeptide and/or BBB modulator peptide (e.g., ADTC5 or HAVN1 peptide) is from about 20% to about 100% of the maximum tolerated dose of each agent. In other embodiments, the suitable dosages of rhApoE2 polypeptide and/or BBB modulator peptide is from about 25% to about 90% of the maximum tolerated dose of each agent. In some embodiments, the suitable dosages of rhApoE2 polypeptide and/or BBB modulator peptide is from about 30% to about 80% of the maximum tolerated dose of each agent. In other embodiments, the suitable dosages of rhApoE2 polypeptide and/or BBB modulator peptide is from about 40% to about 75% of the maximum tolerated dose of each agent. In some embodiments, the suitable dosages of rhApoE2 polypeptide and/or BBB modulator peptide is from about 45% to about 60% of the maximum tolerated dose of each agent. In other embodiments, suitable dosages of rhApoE2 polypeptide and/or BBB modulator peptide is about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose of each agent.

In some embodiments, the rhApoE2 polypeptide and/or BBB modulator peptide is administered one, two, three, four, or five times per day. In other embodiments, the rhApoE2 polypeptide and/or BBB modulator peptide is administered more than five times per day. In some embodiments, the rhApoE2 polypeptide and/or BBB modulator peptide is administered every day, every other day, every third day, every fourth day, every fifth day, or every sixth day. In other embodiments, the rhApoE2 polypeptide and/or BBB modulator peptide is administered weekly, bi-weekly, tri-weekly, or monthly. In some embodiments, the rhApoE2 polypeptide and/or BBB modulator peptide is administered for a period of one, two, three, four, or five weeks. In other embodiments the rhApoE2 polypeptide and/or BBB modulator peptide is administered for six weeks or more. In some embodiments, the rhApoE2 polypeptide and/or BBB modulator peptide is administered for twelve weeks or more. In other embodiments, the rhApoE2 polypeptide and/or BBB modulator peptide is administered for a period of less than one year. In some embodiments, the rhApoE2 polypeptide and/or BBB modulator peptide is administered for a period of more than one year. In other embodiments, the rhApoE2 polypeptide and/or BBB modulator peptide is administered throughout the subject's life.

In some embodiments of the methods of the present technology, the rhApoE2 polypeptide and/or BBB modulator peptide is administered weekly for 1 week or more. In other embodiments, the rhApoE2 polypeptide and/or BBB modulator peptide is administered weekly for 2 weeks or more. In some embodiments, the rhApoE2 polypeptide and/or BBB modulator peptide is administered weekly for 3 weeks or more. In other embodiments, the rhApoE2 polypeptide and/or BBB modulator peptide is administered weekly for 4 weeks or more. In some embodiments, the rhApoE2 polypeptide and/or BBB modulator peptide is administered weekly for 6 weeks or more. In other embodiments, the rhApoE2 polypeptide and/or BBB modulator peptide is administered weekly for 12 weeks or more. In some embodiments, the rhApoE2 polypeptide and/or BBB modulator peptide is administered weekly throughout the subject's life.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Kits of the Present Technology

The present disclosure provides kits for treating a neurodegenerative disease or metabolic disease comprising the plurality of rhApoE2 polypeptides disclosed herein, and instructions for using the same to treat the neurodegenerative disease or metabolic disease. In some embodiments, the kit further comprises a BBB modulator peptide. In certain embodiments, the BBB modulator peptide is the ADTC5 peptide or HAVN1 peptide. When simultaneous administration is contemplated, the kit may comprise the rhApoE2 polypeptide and BBB modulator peptide formulated into a single pharmaceutical composition such as a powder, or as separate pharmaceutical compositions. When the rhApoE2 polypeptide and BBB modulator peptide are not administered simultaneously, the kit may comprise the rhApoE2 polypeptide and BBB modulator peptide formulated as separate pharmaceutical compositions either in a single package, or in separate packages.

The kits may further comprise pharmaceutically acceptable excipients, diluents, or carriers that are compatible with one or more kit components described herein. The kit component compositions may be prepared sterilely, packaged in syringe and shipped refrigerated. Alternatively, it may be housed in a vial or other container for storage. The kit component compositions are optionally premixed. The kit may have one or more or all of the components required to administer the kit component compositions to a patient, such as a syringe or a nasal drug delivery system.

Optionally, the above-described components of the kits of the present technology are packed in suitable containers and labeled for the treatment of neurological diseases including, but not limited to, AD. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way.

### Example 1: Production and Characterization of Physiologically Relevant Recombinant Human ApoE2 (rhApoE2) Sialoglycoprotein

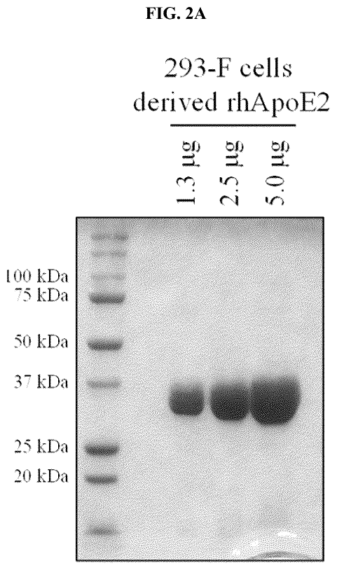

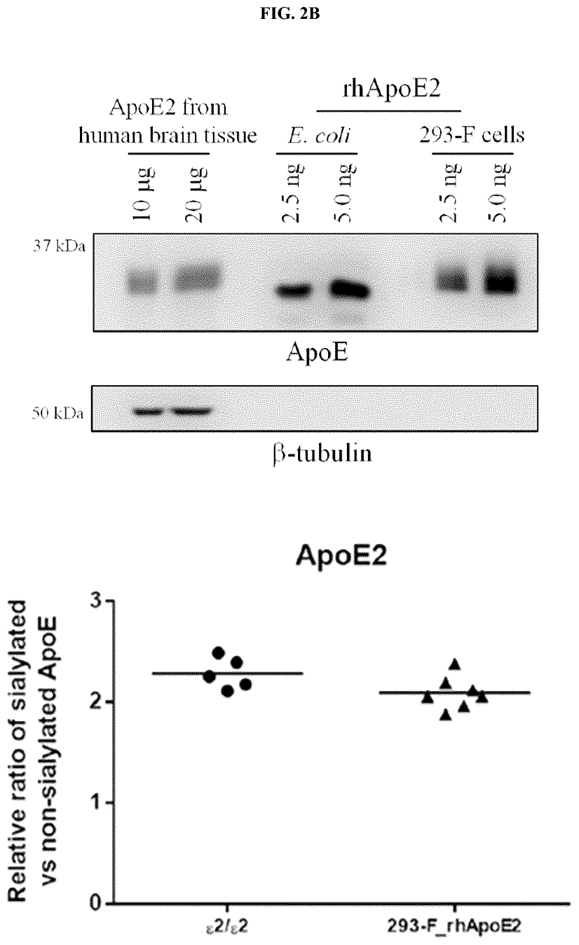

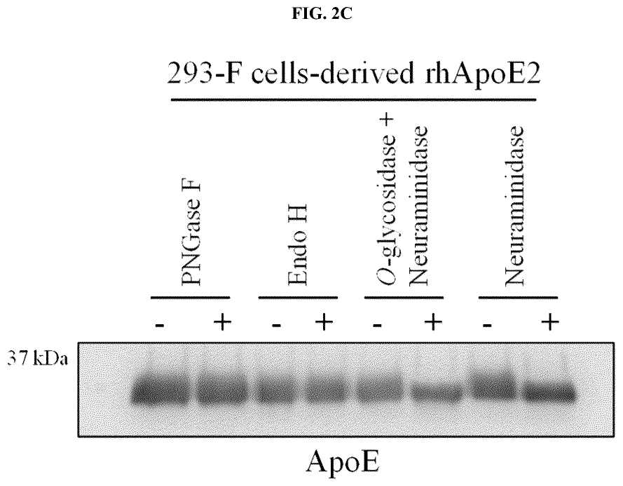

Physiologically relevant recombinant human ApoE2 (rhApoE2) sialoglycoprotein was produced using the FreeStyle 293 expression system. Briefly, FreeStyle 293-F cells were maintained in FreeStyle 293 expression medium and transfected with pcDNA3.1 (−)-hApoE2-Strep II (a mammalian expression vector for human ApoE2 with the C-terminus fused with a Strep II-tag) using 293fectin transfection reagent. The culture medium was collected, concentrated, and purified using a gravity flow Strep-Tactin superflow column to obtain pure rhApoE2 (>95% purity) (FIG. 2A). Nearly identical to the ApoE2 expressed in the human cortex, rhApoE2 derived from 293-F cells presents two prominent bands indicating posttranslational modification, which is absent in rhApoE2 derived from E. coli. The band intensity ratio of sialo_ApoE2 versus asialo_ApoE2 in 293-F cells-derived rhApoE2 was not significantly different from ApoE2 in human brain tissue, further demonstrating the compatibility of 293-F-rhApoE2 with the human form of ApoE2 (FIG. 2B). Moreover, treatment with deglycosylation enzymes that specifically target N-linked glycans (PNGase F and Endo H) did not alter the ApoE immunoreactive bands, indicating the lack of N-linked glycans in 293-F-rhApoE2. As expected, treatment with neuraminidase alone or in combination with O-glycosidase resulted in the disappearance of the upper band, indicating that 293-F-rhApoE2 is a sialic acid-enriched protein (FIG. 2C). Collectively, these data confirm that rhApoE2 derived from 293-F cells is post-translationally modified by O-linked glycosylation and sialylation; these structural features are consistent with physiological patterns of ApoE2 protein expressed in the human brain.

rhApoE2 protein sequence (SEQ ID NO: 1):
KVEQAVETEPEPELRQQTEWQSGQRWELALGRFWDYLRWVQTLSEQVQE
ELLSSQVTQELRALMDETMKELKAYKSELEEQLTPVAEETRARLSKELQ
AAQARLGADMEDVCGRLVQYRGEVQAMLGQSTEELRVRLASHLRKLRKR
LLRDADDLQKCLAVYQAGAREGAERGLSAIRERLGPLVEQGRVRAATVG
SLAGQPLQERAQAWGERLRARMEEMGSRTRDRLDEVKEQVAEVRAKLEE
QAQQIRLQAEAFQARLKSWFEPLVEDMQRQWAGLVEKVQAAVGTSAAPV
PSDNH

### Example 2: rhApoE2 Treatment Upregulates Akt Activity and HK2 Protein Expression and Downregulates Endogenous Levels of ApoE4 in ApoE4-Expressing Primary Cortical Neurons

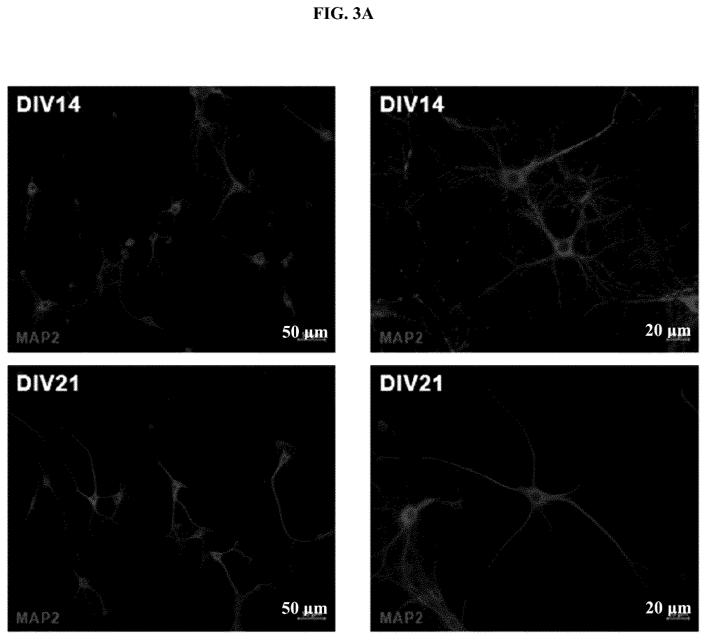

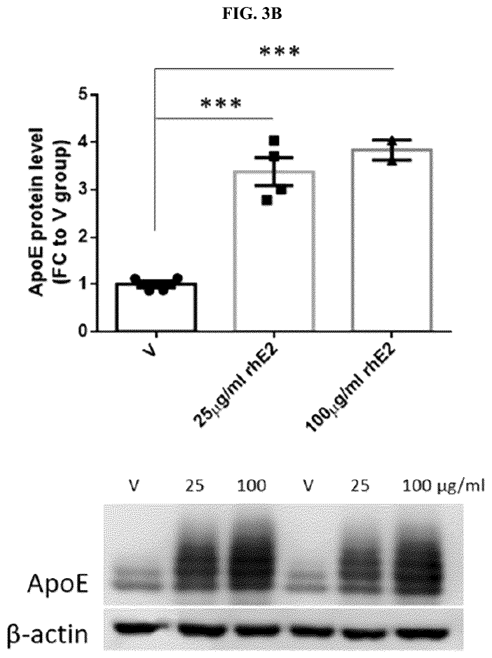

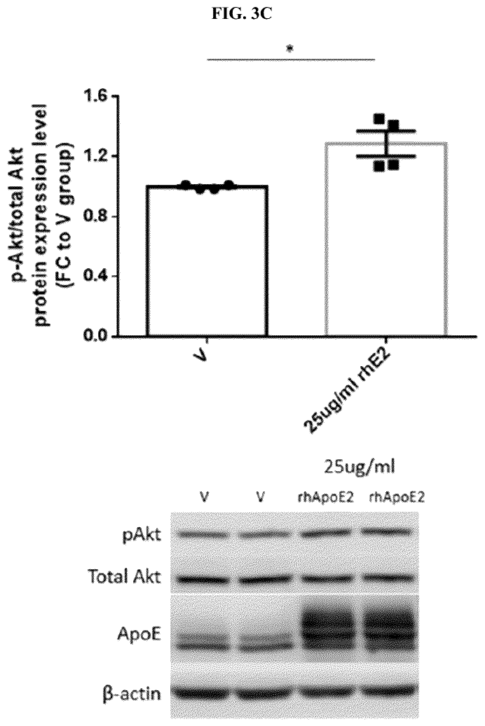

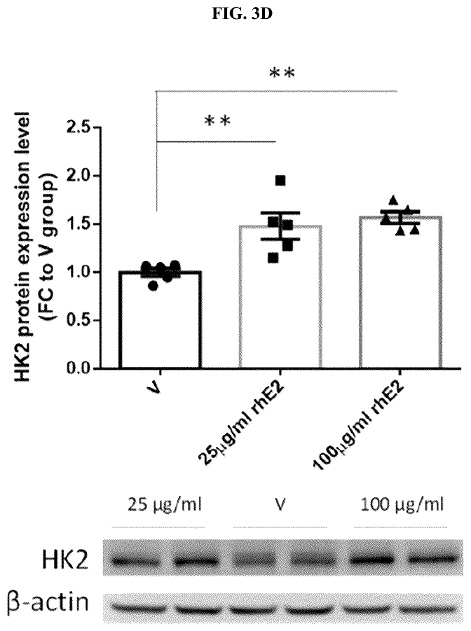

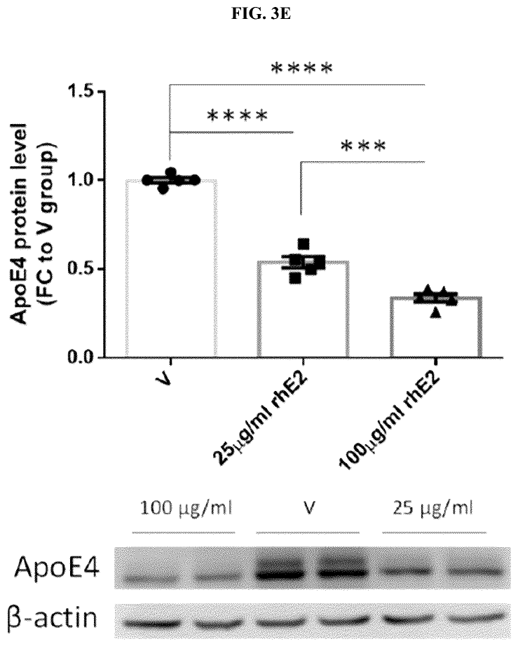

To determine if exposure to rhApoE2 elicit changes in neuronal metabolic activity in ApoE4-expressing neurons, we investigated this question using primary cortical neuronal cell models prepared from human ApoE4 knockin (hApoE4KI) postnatal day 0-2 (PO-2) mouse brains. The maturity of neuronal cultures at 14 or 21 days in vitro (DIV) was validated by the observation of neuronal extensions and expression of neuronal and synaptic protein markers (FIG. 3A). Neurons were treated with 25 μg/ml or 100 μg/ml rhApoE2, or vehicle alone, for 1-2 days; 100 μg/ml rhApoE2 corresponds to the physiological level of ApoE2 in human serum.¹⁰⁷ When compared to neurons exposed to vehicle alone, neurons exposed to rhApoE2 exhibited significant uptake of rhApoE2 (FIG. 3B), increased Akt phosphorylation (FIG. 3C), increased HK2 protein expression (FIG. 3D), and decreased endogenous levels of ApoE4 (FIG. 3E). Without wishing to be bound by theory, it is believed that rhApoE2 has the therapeutic potential to promote neuronal health and resilience through upregulating PI3K/Akt signaling cascades and HK2-mediated metabolic processes, and also by ameliorating the neurodegenerative impact of ApoE4 in ApoE4-expressing neurons.

### Example 3: rhApoE2 Treatment Promotes Neuronal Viability and Protein Homeostasis Against Neurotoxic Insults in ApoE4-Expressing Primary Cortical Neurons

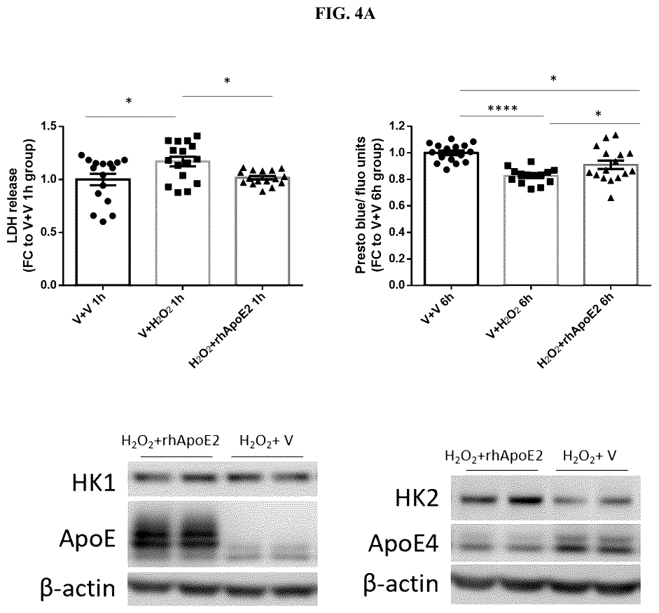

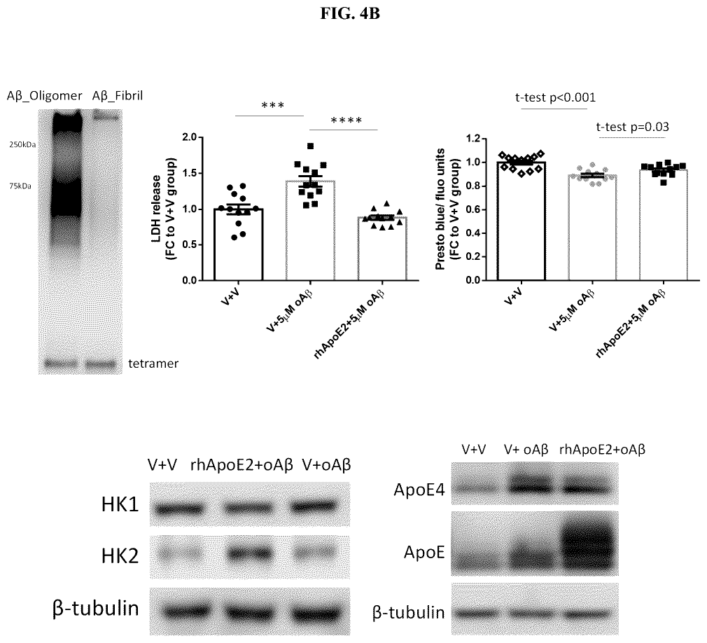

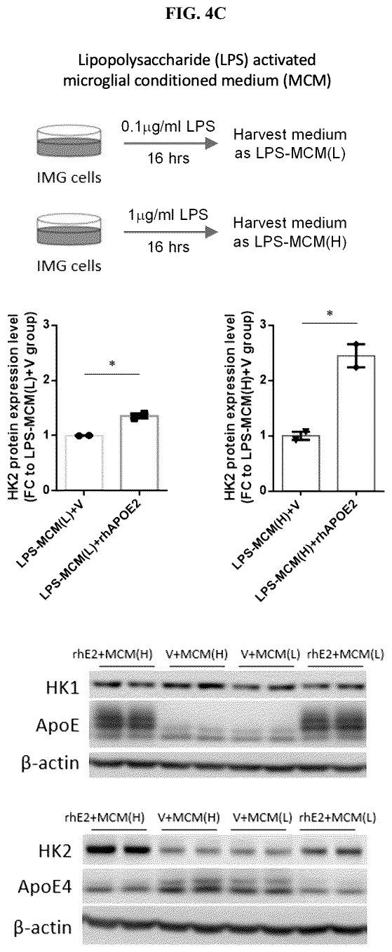

rhApoE2 treatment promoted neuronal viability and attenuated the adverse impact on neuronal expression of HK2 and ApoE4 induced by neurotoxic insults in ApoE4-expressing primary cortical neurons. The therapeutic potential of rhApoE2 was further evaluated in ApoE4-expressing primary cortical neurons in response to neurotoxic insults, including H₂O₂, oligomeric amyloid β (oAβ), and conditioned medium collected from lipopolysaccharide (LPS)-treated microglia, or MCM. For the H₂O₂ challenge, hApoE4KI neurons were pretreated with 25 μg/ml rhApoE2 or vehicle alone for 2 days and then exposed to 100 μM H₂O₂ for 30 minutes, followed by 1 or 6 additional hours of incubation with rhApoE2 or vehicle alone. Neurons pretreated with rhApoE2 presented significantly better outcomes on measures of neuronal membrane integrity and metabolic activity, indicating improved viability compared to neurons pretreated with vehicle alone in responding to H₂O₂-induced neurotoxicity (FIG. 4A). Moreover, rhApoE2 increased neuronal HK2 protein expression and decreased endogenous ApoE4 compared to vehicle alone-treated neurons. For the oAβ challenge, hApoE4KI neurons were pretreated with 25 μg/ml rhApoE2 or vehicle alone for 2 days and then exposed to 5 μM oAβ for 2 additional days. Similarly, the rhApoE2 treatment promoted neuronal viability and HK2 protein expression and attenuated the induction of ApoE4 caused by oAβ-mediated neurotoxicity (FIG. 4B). To evaluate the impact of rhApoE2 on neuronal responses to microglial inflammation, immortalized microglial (IMG) cells were stimulated with 0.1 μg/ml (low dose) or 1 μg/ml (high dose) LPS. After 16 hours of exposure to LPS, LPS-MCM were collected and diluted at 1:200 into neuronal culture medium. hApoE4KI neurons were challenged by LPS-MCM with or without 25 μg/ml rhApoE2 for 2 days. Consistent with the above results, neurons exposed to rhApoE2 expressed significantly higher levels of HK2 and lower levels of ApoE4 compared to neurons exposed to vehicle alone in response to both low and high LPS-MCM (FIG. 4C). Taken together, these results support a neuroprotective role of rhApoE2 against neurodegenerative risks.

### Example 4: ADTC5 Peptide Improves Brain Delivery of rhApoE2 Via Tail Vein Injection in hApoE4KI Mice

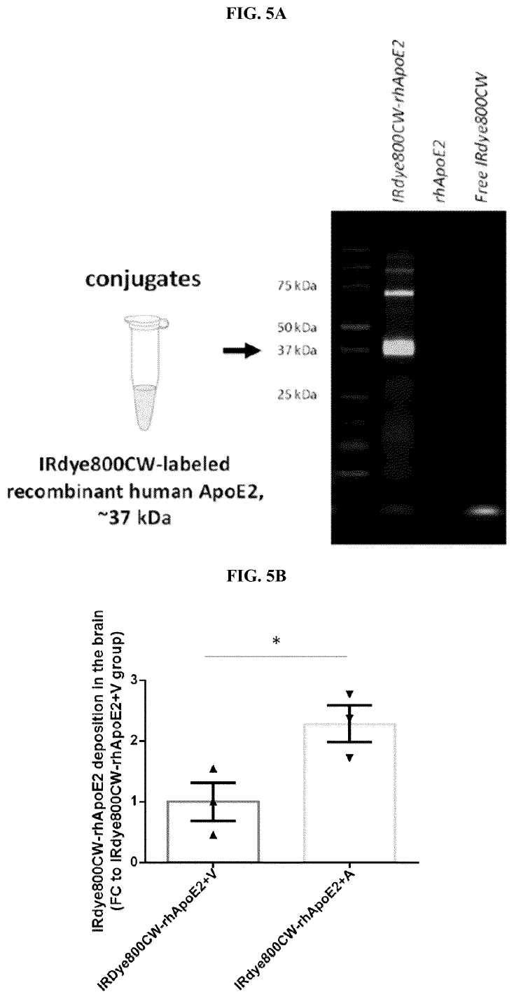

A major hurdle in the development of protein therapies for treating brain diseases is the difficulty of delivering therapies across the blood-brain barrier (BBB). To address this challenge, a noninvasive approach was used involving modulation of cadherin-cadherin interactions on the BBB, thereby increasing the porosity of the paracellular pathway of brain transport. Cadherin peptides have been demonstrated to improve the delivery of proteins up to 150 kDa into the brains of living mice and rats safely and effectively, with ADTC5 shown as one of the most effective modulator when administered intravenously.¹⁰⁸⁻¹¹³ rhApoE2 has a molecular size of 34-39 kDa. A conjugate of rhApoE2 with IRdye800CW (producing near-infrared fluorescence [NIRF]) was constructed, which provided a sensitive imaging tool for detection of brain deposition of rhApoE2 following administration. The IRdye800CW-labeled rhApoE2 was purified using Pierce Zeba desalting spin columns. The purity of the conjugated rhApoE2 was analyzed and verified by SDS-PAGE followed by Western blot analyses (FIG. 5A). The purified IRdye800CW-labeled rhApoE2 with or without ADTC5 was then administered via tail vein injection to 4-6-months-old female and male hApoE4KI mice. 40-60 minutes post-injection, mice were anesthetized and transcardial perfusion was performed with PBS+Tween-20 to eliminate residual dye and IRdye-rhApoE2 in brain microvessels to minimize false-positive effects. NIRF imaging data revealed that co-administration of IRdye800CW-rhApoE2 with ADTC5 resulted in a significant deposition of IRdye800CW-rhApoE2 throughout the brain; by contrast, brain distribution of IRdye800CW-rhApoE2 when administered without ADTC5 was nearly non-detectable (FIGS. 5B-5C). In conclusion, rhApoE2 was successfully delivered along with ADTC5 into the brains of hApoE4KI mice via tail vein injection.

### Example 5: Intravenous Administration of rhApoE2 Upregulates Cortical HK2 Expression and Activity and Potentiates Synaptic Vesicle Exocytosis in Response to Depolarization in Mid-Aged hApoE4KI Mice

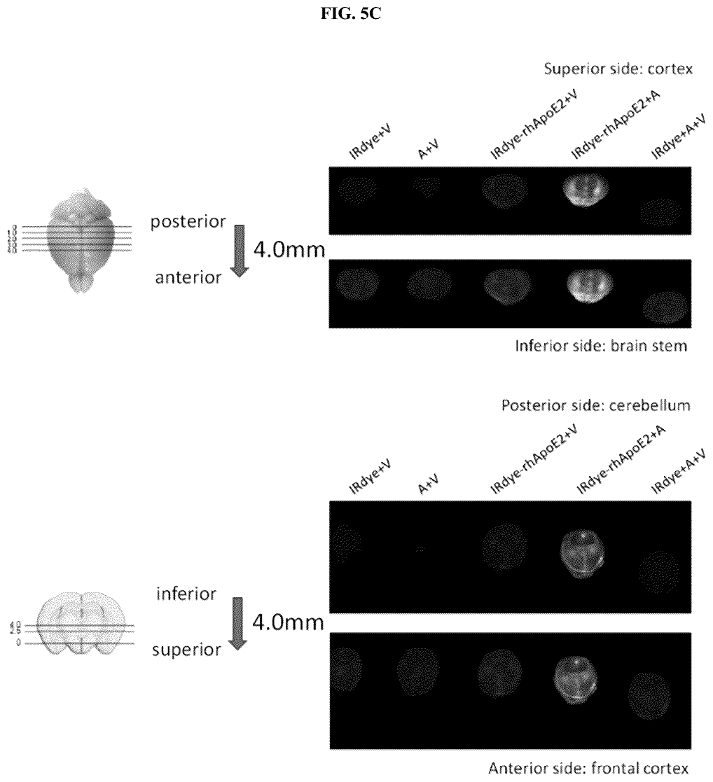

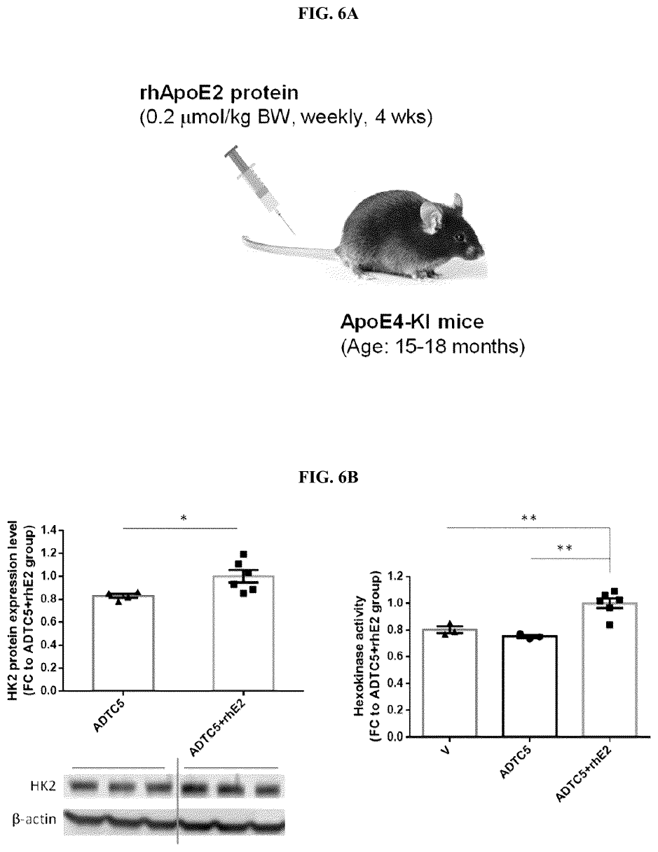

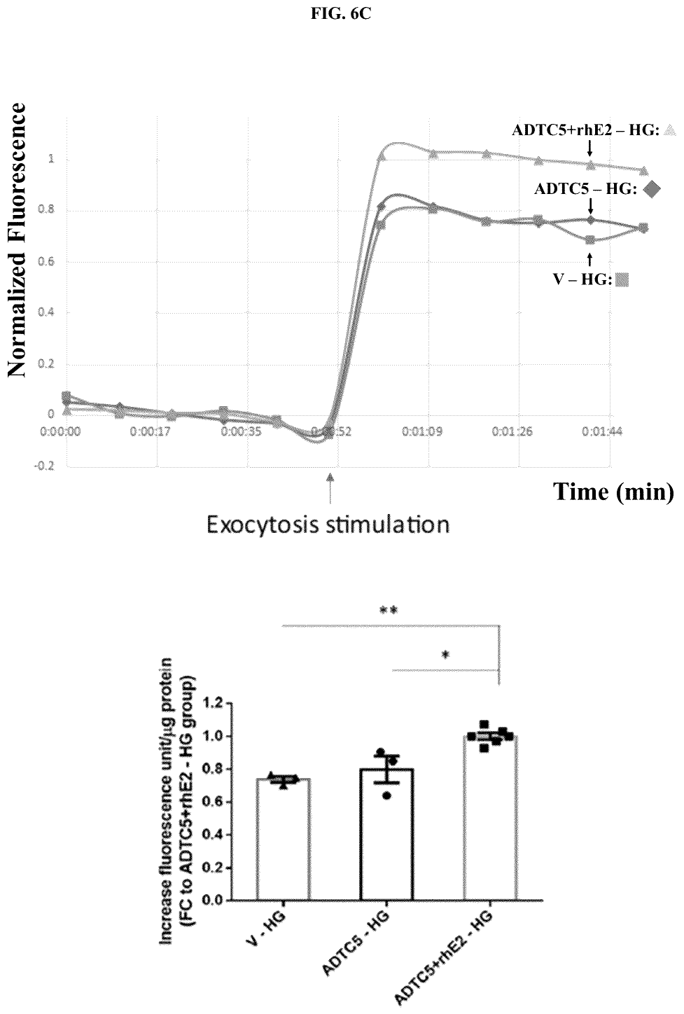

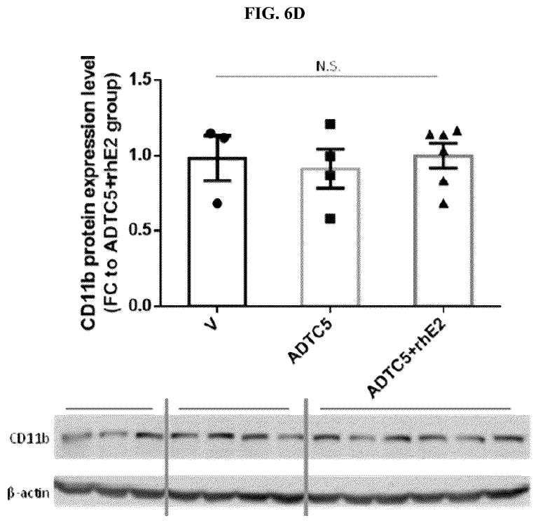

To evaluate the therapeutic impact of rhApoE2 delivery on brain changes associated with AD, 14-18-month-old hApoE3KI and hApoE4KI mice of both sexes were intravenously administered pure, non-conjugated rhApoE2 (0.2 μmol/kg BW) along with ADTC5 (10 μmol/kg BW), rhApoE2 with vehicle alone, or ADTC5 alone, via tail vein injection, once weekly, for 4 weeks. For the rhApoE2 dose, 0.2 μmol/kg BW was chosen based on literature that indicates ApoE is present in the blood at an average level of 5.5-14.5 mg/dl in both human and mice.^{107,114,115} Given that the average weight of the mice was 38 g, a mean blood volume of 77-80 μl/g, and the molecular weight of rhApoE2, 0.2 μmol/kg BW rhApoE2 corresponds to approximately 8 mg/dl in the blood, which is within the physiological range. Brains were harvested 24 hours after the last injection, and cortical tissues were analyzed in subsequent experiments. HK2 expression and activity were significantly upregulated in cortical tissues of hApoE4KI mice treated with rhApoE2 along with ADTC5, compared to mice treated with rhApoE2 along with vehicle, or ADTC5 alone (FIGS. 6A-6B). To evaluate the functional impact of rhApoE2, synaptosomes were isolated from freshly harvested cortical tissues of treated mice. Synaptic vesicle release activity was measured in response to KCl (60 mM)-induced neuronal depolarization by changes in acridine orange (AO) fluorescence. AO fluorescence units were normalized by total protein contents in synaptosomes. Mice treated with rhApoE2 along with ADTC5 exhibited significantly heightened synaptic vesicle exocytosis compared to the other two control groups, indicating a more robust synaptic activity promoted by rhApoE2 (FIG. 6C). Moreover, analyses of brain homogenates of treated mice showed no sign of toxicity, as indicated by the absence of astrogliosis and microglial inflammation as measured by CD11b expression (FIG. 6D). Without wishing to be bound by theory, it is believed that these results demonstrate that brain delivery of rhApoE2 has the potential to strengthen synaptic function effectively and safely in part through upregulation of HK2-mediated glycolytic activity.

### Example 6: Intravenous Administration of rhApoE2 Promotes Cortical HK Activity, Protein Homeostasis, and Spatial Learning and Memory Function in Aged hApoE4KI Mice

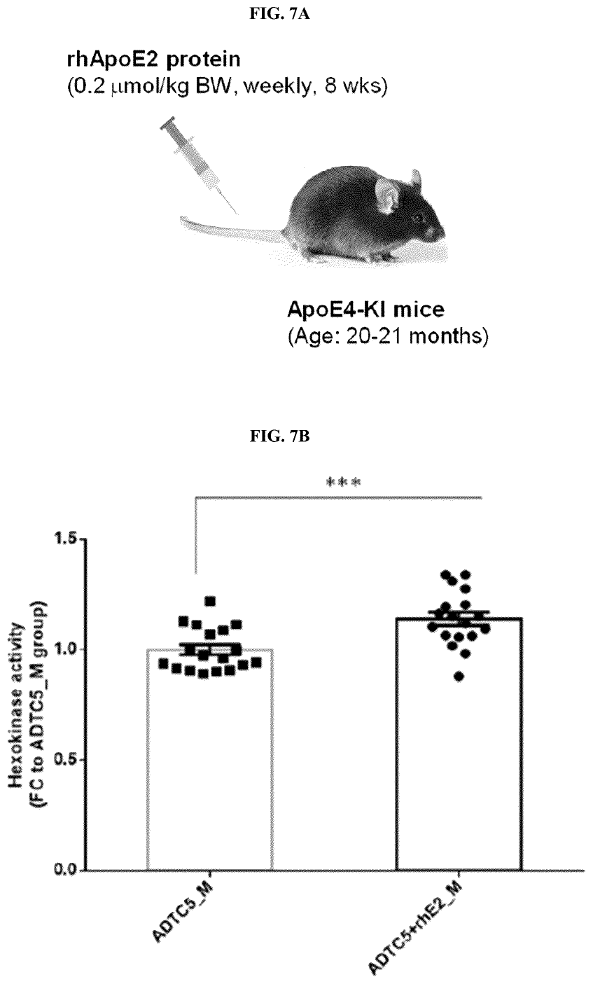

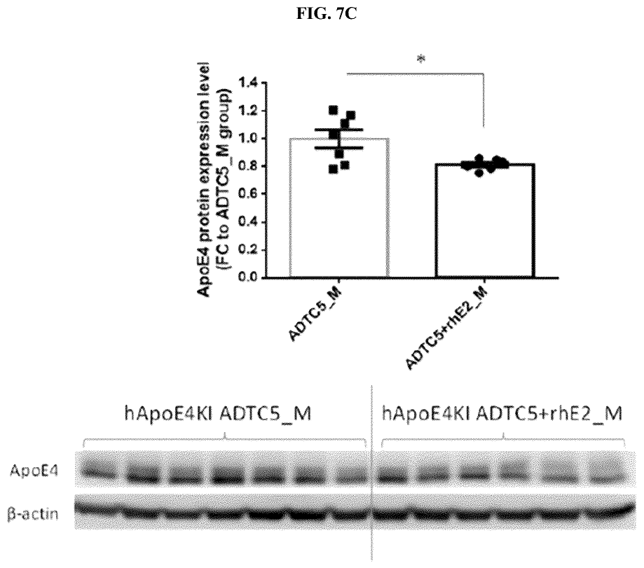

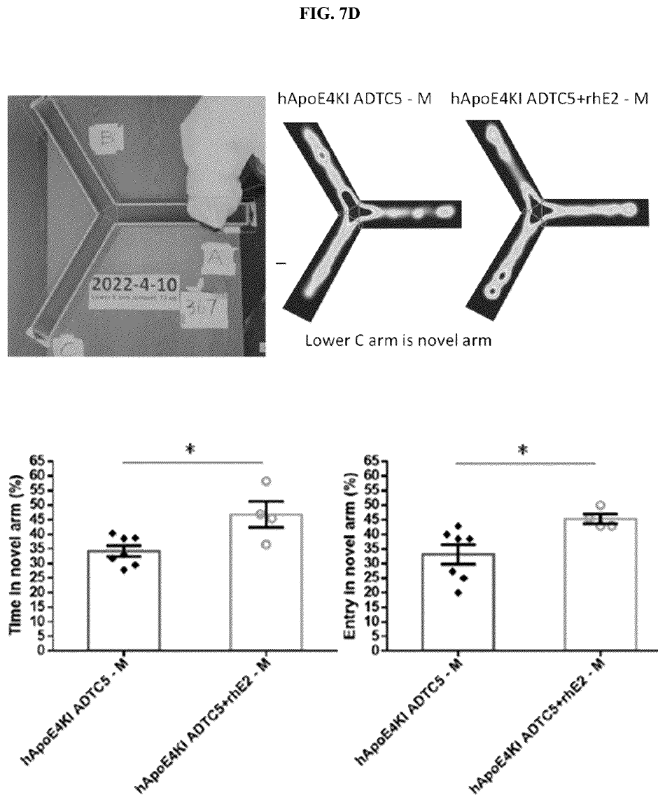

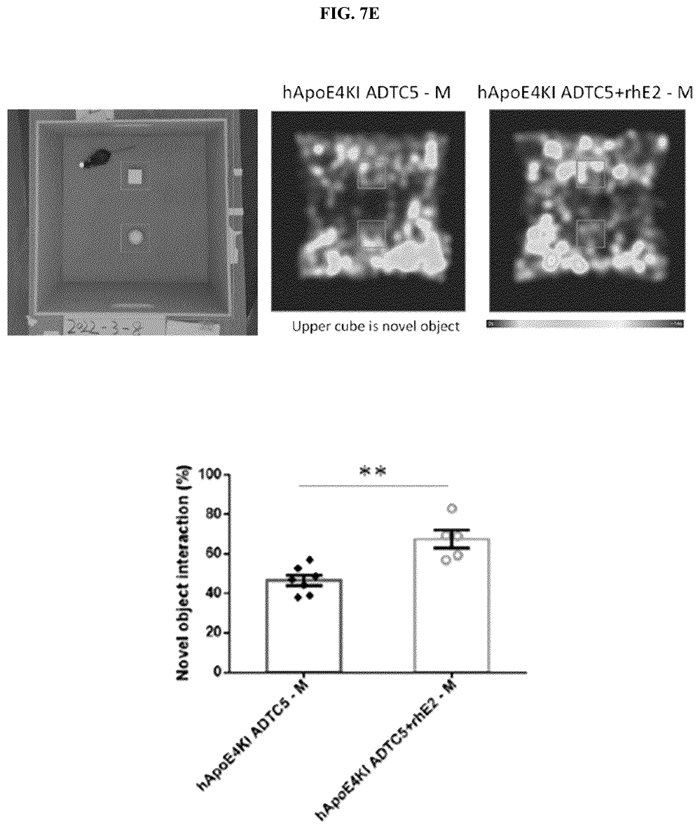

To further demonstrate the therapeutic effects of rhApoE2 on the brain, a two-month study in aged (20-21-month-old) hApoE3KI and hApoE4KI mice of both sexes was performed. Mice were treated with intravenous administration of pure, non-conjugated rhApoE2 (0.2 μmol/kg BW) along with ADTC5 (10 μmol/kg BW), rhApoE2 with vehicle alone, or ADTC5 alone, via tail vein injection, once weekly, for 8 weeks (FIG. 7A). Two weeks before the last injection, mice were subjected to cognition behavioral evaluation of spatial learning and memory function in Y-maze two-trial test and novel object recognition test. Brains were harvested 1-3 days after the last injection and cortical tissues were analyzed in subsequent experiments. Brain delivery of rhApoE2 promoted cortical HK activity (FIG. 7B), attenuated endogenous ApoE4 (FIG. 7C), and improved the cognitive performance of mice in both Y-maze two-trial test (FIG. 7D) and novel object recognition test (FIG. 7E). Notably, these effects were more prominent in male mice than female mice, indicating that sex may play a role in modulating the therapeutic responses to rhApoE2. These results further demonstrate that brain delivery of rhApoE2 can have a beneficial impact on cognitive function and associated metabolic pathways.

### Example 7: Intravenous Administration of rhApoE2 Improves Blood Lipid Profile and Reduces Splenic Expression of Inflammatory Markers in Aged hApoE4KI Mice

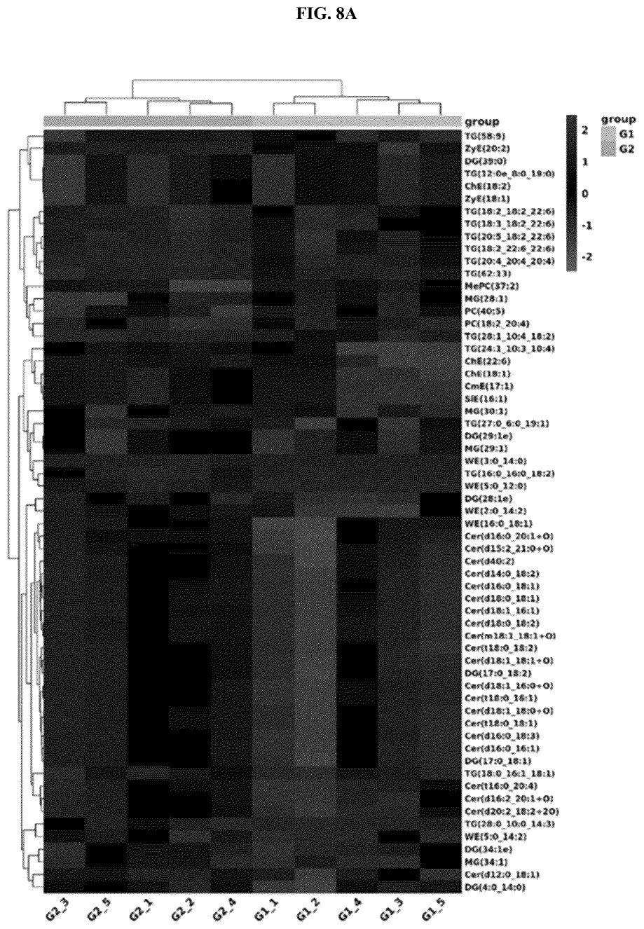

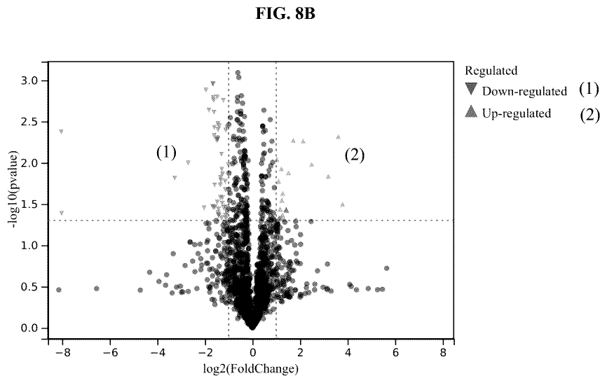

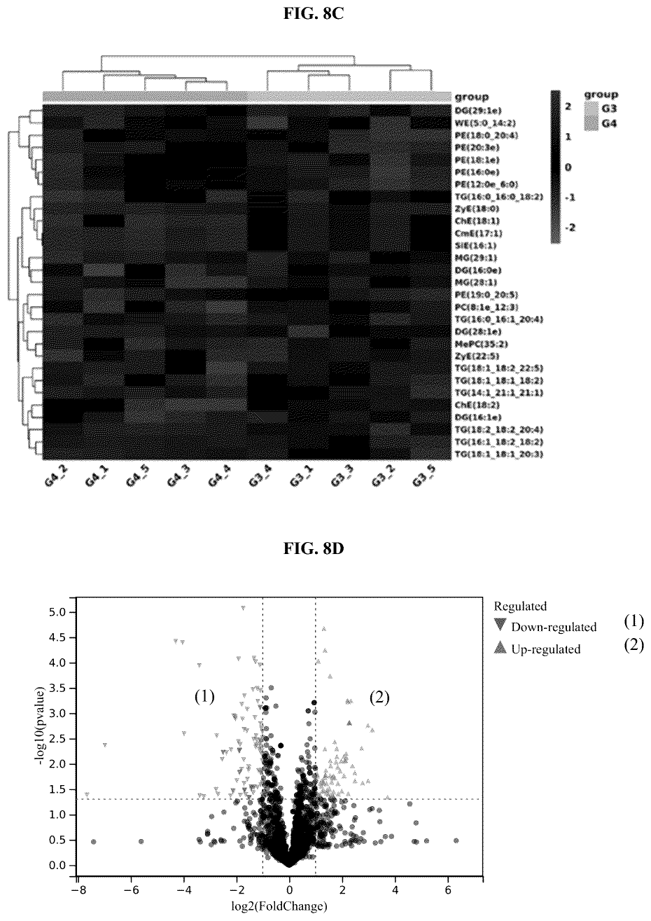

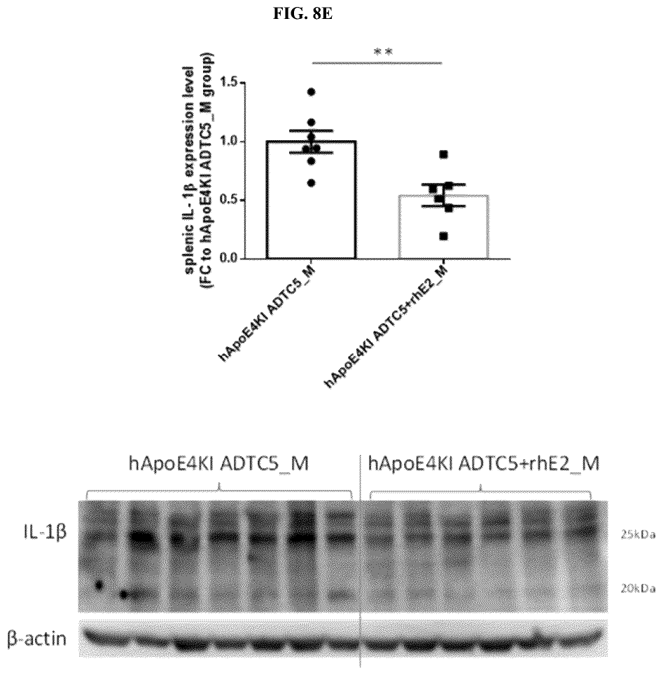

Given that ApoE plays a major role in lipid metabolism, a lipidomics profiling analysis was performed on serum samples collected from aged hApoE4KI mice subjected to the two-month treatment with 8 intravenous injections, once weekly, of rhApoE2 or vehicle alone. The lipidomics results revealed that the rhApoE2 treatment substantially reprogrammed the circulating lipidome in aged ApoE4-KI mice in a sex-dependent manner. Specifically, rhApoE2 significantly altered the levels of 17 ceramides in the serum of male mice, and they were all downregulations. Moreover, in male mice serum, rhApoE2 decreased the levels of triglycerides (TGs) that contain saturated fatty acids (SFAs) or monounsaturated fatty acids (MUFAs), whereas it increased the levels of TGs that contain long-chain polyunsaturated fatty acids (PUFAs), including alpha-linolenic acid, eicosapentaenoic acid (EPA), and docosahexaenoic acid (DHA) (FIGS. 8A-8B, Table 1, Table 2). In female mice, rhApoE2 similarly upregulated the serum levels of long-chain-PUFAs-containing TGs, in addition to downregulating several phosphatidylethanolamines (FIGS. 8C-8D, Table 1, Table 3). High blood ceramide levels have been shown to predict cognitive decline and increased risk of AD.¹¹⁶⁻¹¹⁹ In addition, studies conducted in rodents revealed that inhibition of ceramide biosynthesis or increasing ceramide degradation substantially reduced insulin resistance, inflammation, mitochondrial stress, and improved systemic metabolism¹²⁰⁻¹²³ Without wishing to be bound by theory, it is believed that these studies suggest that reduced levels of ceramides in the blood may contribute to the reduced expression of inflammatory markers in the spleen of mice treated with rhApoE2 (FIG. 8E).

Long-chain PUFAs, such as EPA and DHA, are widely regarded to be lipoprotective and promote healthy aging throughout life.^{124,125} The brain is particularly enriched with EPA and DHA, which are found in abundance in neuronal membranes and play vital roles in neuronal maturation, neurite outgrowth, and neurotransmitter release.¹²⁶⁻¹³⁰ EPA and DHA have also been broadly indicated for their beneficial effects in the aging brain, including maintaining white matter microstructural integrity and strengthening the neural circuits that are vulnerable to age-related decline.¹³¹⁻¹³⁵ In older adults, lower levels of DHA in the blood have been associated with smaller brain volumes, a marker of accelerated brain aging, and increased incidence of AD.^{136,137} Consistent with the neuroprotective properties of PUFAs in the brain, a recent study of 689 participants from the Alzheimer's Disease Neuroimaging Initiative cohort revealed a significant association between long-chain-PUFAs-containing TGs (PUTGs) and mild cognitive impairment (MCI) and AD. Lower serum levels of PUTGs were observed and associated with increased brain atrophy and decreased CSF amyloid-beta concentrations in individuals with MCI and AD compared to cognitively normal controls, and these associations were more pronounced in ApoE4 carriers.¹³⁸ Without wishing to be bound by theory, it is believed that the peripheral benefits elicited by rhApoE2, as demonstrated here, including improvement of circulating lipid profile and reduction of systemic inflammation, may contribute to its neuroprotective roles and therapeutic potential in treating or preventing neurodegenerative diseases such as AD.

TABLE 1
upregulated and downregulated neutral lipids, sphingolipids,
and phospholipids in hApoE4KI mice treated with rhApoE2.

| | male rhApoE2 + ADTC5 (G1) | | |
|---|---|---|---|
| | versus | | |
| | male ADTC5 control (G2) | | |
| Lipid class | FC(G1/G2) | T-Test (p) | VIP |

Neutral lipids

| | | | |
|---|---|---|---|
| TG(62:14) | 5.602 | 0.010 | 1.766 |
| TG(62:13) | 4.372 | 0.005 | 1.855 |
| TG(60:13) | 9.089 | 0.014 | 1.714 |
| TG(60:12) | 3.281 | 0.005 | 1.857 |
| TG(58:9) | 2.405 | 0.043 | 1.501 |
| TG(58:11) | 2.139 | 0.017 | 1.689 |
| TG(58:10) | 2.043 | 0.009 | 1.785 |
| TG(56:7) | 12.179 | 0.005 | 1.870 |
| TG(39:0e) | 2.698 | 0.038 | 1.531 |

Sphingolipids

| | | | |
|---|---|---|---|
| Cer(d30:1) | 0.333 | 0.019 | 1.672 |
| Cer(d32:1) | 0.362 | 0.005 | 1.864 |
| Cer(d32:2) | 0.398 | 0.016 | 1.693 |
| Cer(d34:1) | 0.393 | 0.015 | 1.707 |
| Cer(d34:1 + O) | 0.323 | 0.002 | 1.974 |
| Cer(d34:2) | 0.417 | 0.027 | 1.603 |
| Cer(d34:3) | 0.326 | 0.005 | 1.873 |
| Cer(d36:1) | 0.454 | 0.021 | 1.647 |
| Cer(d36:1 + O) | 0.461 | 0.004 | 1.890 |
| Cer(d36:1 + O) | 0.362 | 0.005 | 1.859 |
| Cer(d36:2) | 0.468 | 0.038 | 1.529 |
| Cer(d36:2 + O) | 0.494 | 0.011 | 1.764 |
| Cer(d36:2 + O) | 0.368 | 0.004 | 1.892 |
| Cer(d36:3 + O) | 0.327 | 0.002 | 1.943 |
| Cer(d38:4 + 2O) | 0.358 | 0.002 | 1.983 |
| Cer(d40:2) | 0.452 | 0.017 | 1.689 |
| Cer(m36:2 + O) | 0.461 | 0.035 | 1.549 |
| Cer(t34:1) | 0.318 | 0.002 | 1.982 |
| Cer(t36:1) | 0.362 | 0.005 | 1.859 |
| Cer(t36:2) | 0.329 | 0.004 | 1.898 |
| Cer(t36:4) | 0.352 | 0.005 | 1.857 |

Neutral lipids

| | | | |
|---|---|---|---|
| DG(18:0) | 0.429 | 0.002 | 1.975 |
| DG(28:1e) | 0.334 | 0.035 | 1.550 |
| DG(29:1e) | 0.324 | 0.035 | 1.548 |
| DG(34:1e) | 0.433 | 0.035 | 1.545 |
| DG(35:1) | 0.379 | 0.004 | 1.903 |
| DG(35:2) | 0.365 | 0.003 | 1.911 |
| TG(52:3) | 0.153 | 0.010 | 1.771 |
| TG(52:2) | 0.410 | 0.012 | 1.744 |
| TG(52:1) | 0.492 | 0.038 | 1.528 |
| TG(50:2) | 0.104 | 0.015 | 1.705 |
| TG(44:8) | 0.394 | 0.026 | 1.606 |

| | female rhApoE2 + ADTC5 (G3) | | |
|---|---|---|---|
| | versus | | |
| | female ADTC5 control (G4) | | |
| Lipid class | FC(G3/G4) | T-Test (p) | VIP |

Neutral lipids

| | | | |
|---|---|---|---|
| TG(58:8) | 2.500 | 0.018 | 2.234 |
| TG(56:8) | 3.757 | 0.019 | 2.222 |
| TG(56:5) | 5.077 | 0.029 | 2.107 |
| TG(56:3) | 4.581 | 0.027 | 2.132 |
| TG(54:4) | 3.182 | 0.031 | 2.090 |
| TG(52:5) | 6.401 | 0.041 | 2.015 |
| TG(52:5) | 4.091 | 0.020 | 2.203 |
| TG(50:2) | 2.321 | 0.016 | 2.255 |

Phospholipids

| | | | |
|---|---|---|---|
| PE(16:0e) | 0.373 | 0.004 | 2.534 |
| PE(18:0e) | 0.370 | 0.003 | 2.544 |
| PE(18:1e) | 0.409 | 0.021 | 2.195 |
| PE(20:3e) | 0.374 | 0.036 | 2.048 |
| PE(38:4) | 0.352 | 0.022 | 2.181 |

TABLE 2
upregulated and downregulated triglycerides in hApoE4KI male mice
treated with rhApoE2.

| | Standardized name | | |
|---|---|---|---|
| Fatty acids | FA1 | FA2 | FA3 |

Upregulated triglycerides in hApoE4KI mole mice treated with thApoE2

| | | | | |
|---|---|---|---|---|
| TG(62:14) | (18:2_22:6_22:6) | Linoleic acid | DHA | DHA |
| TG(62:13) | (62:13) | | | |
| TG(60:13) | (20:5_18:2_22:6) | Eicosapentaenoic acid | Linoleic acid | DRA |
| TG(60:12) | (20:4_20:4_20:4) | Arachidonic add | Arachidonic acid | Arachidonic acid |
| TG(58:9) | (58:9) | | | |
| TG(58:11) | (18:3_18:2_22:6) | Octadecatrienoic acid | Linoleic acid | DHA |
| TG(58:10) | (18:2_18:2_22:6) | Linoleic acid | Linoleic acid | DHA |
| TG(56:7) | (28:1_10:4_18:2) | Octacosenoic acid | | Linoleic acid |
| TG(39:0e) | (12:0e_8:0_19:0) | | Caprylic acid | Nonadecylic acid |

Downregulated triglycerides in hApoE4KI male mice treated with rhApoE2

| | | | | |
|---|---|---|---|---|
| TG(52:1) | (27:0_6:0_19:1) | Carboceric acid | Caproic acid | Nonadecenoic acid |
| TG(52:3) | (28:0_10:0_14:3) | Montanic acid | Capric acid | Tetradecatrienoic acid |
| TG(52:2) | (18:0_16:1_18:1) | Stearic acid | Hexadecenoic acid | Qctadecenoic acid |
| TG(50:2) | (16:0_16:0_18:2) | Palmitic acid | Palmitic acid | Linoleic acid |
| TG(44:8) | (24:1_10:3_10:4) | Tetracosenoic acid | Decatrienoic acid | |

TABLE 3
upregulated triglycerides in hApoE4KI female mice treated with rhApoE2.
Upregulated triglycerides in hApoE4KI female mice treated with rhApoE2

| | | Standardized name | | |
|---|---|---|---|---|
| | Fatty acids | FA1 | FA2 | FA3 |
| TG(58:8) | (18:1_18:2_22:5) | Octadecenoic acid | Linoleic acid | Docosapentaenoic acid |
| TG(56:8) | (18:2_18:2_20:4) | Linoleic acid | Linoleic acid | Arachidonic acid |
| TG(56:5) | (18:1_18:1_20:3) | Octadecenoic acid | Octadecenoic acid | Eicosatrienoic acid |
| TG(56:3) | (14:1_21:1_21:1) | Myristoleic acid | Heneicosenoic acid | Heneicosenoic acid |
| TG(54:4) | (18:1_18:1_18:2) | Octadecenoic acid | Octadecenoic acid | Linoleic acid |
| TG(52:5) | (16:1_18:2_18:2) | Hexadecenoic acid | Linoleic acid | Linoleic acid |
| TG(52:5) | (16:0_16:1_20:4) | Palmitic acid | Hexadecenoic acid | Arachidonic acid |
| TG(50:2) | (16:0_16:0_18:2) | Palmitic acid | Palmitic acid | Linoleic acid |

### Example 8: Roles of Ceramides and Triglycerides in Metabolic Syndrome and AD

In the periphery, ApoE plays a major role in mediating the clearance of TG-rich lipoproteins through ApoE receptors such as low-density lipoprotein receptor (LDLR)-medicated endocytosis in hepatocytes. ApoE2 homozygosity has been associated with an increased risk for cardiovascular pathologies attributed in large part to the low binding affinity of ApoE2 to LDLR relative to ApoE3 and ApoE4, leading to increased lipid accumulation in the circulation.¹⁴⁰ However, hyperlipidemia is only observed in 5-10% of ApoE2 homozygotes, suggesting that the ApoE2 genotype alone is not sufficient to cause this risk phenotype.¹⁴¹ In fact, most ApoE2 homozygotes are found to be normolipidemic or hypolipidemic, and studies have shown the impact of other genes or hormones in converting the hypolipidemia to hyperlipidemia.^{142, 143} Based on these prior reports, it was predicted that introduction of rhApoE2 into the circulation was unlikely to induce a significant hyperlipidemic risk profile.

To prove this prediction, a lipidomics profiling analysis on serum samples collected from 20-21-month-old human ApoE4 knock-in (ApoE4-KI) mice subjected to a two-month treatment with 8 intravenous injections, once weekly, of rhApoE2 at a physiologically relevant dosage (10 μg/g [BW]), or vehicle alone was performed. The lipidomics results revealed that the rhApoE2 treatment substantially reprogramed the circulating lipidome in aged ApoE4-KI mice in a sex-dependent manner (FIGS. 8A-8B and Tables 4-5). The study herein exhibited a remarkedly downregulated diacylglycerols (DAG) by rhApoE2, suggesting the therapeutic role conveyed by rhApoE2 treatment. Notably, the reduced DAG and TAG species were less unsaturated than the upregulated TAG.

Specifically, rhApoE2 significantly downregulated the levels of 17 ceramides in the serum of male ApoE4-KI mice. Moreover, in male mice serum, rhApoE2 decreased the levels of TGs that contain SFAs or MUFAs, whereas it increased the levels of TGs that contain long-chain PUFAs, including alpha-linolenic acid, EPA, and DHA. In addition, a great proportion of DHA- and linoleic acid (LA)-carrying PUTGs was upregulated in rhApoE2-treated male mice and the reduced PUTGs in male mice appeared to be shorter chains and with less degree of unsaturation. In female mice, rhApoE2 similarly upregulated the serum levels of long-chain-PUFAs-containing TAGs, in addition to downregulating several phosphatidylethanolamines (PE). In summary, our lipidomics profiling data indicate that intravenous rhApoE2 administration can improve the serum lipid profile in hApoE4KI mice by increasing long-chain PUTGs and reducing levels of ceramides, DAG, and PE.

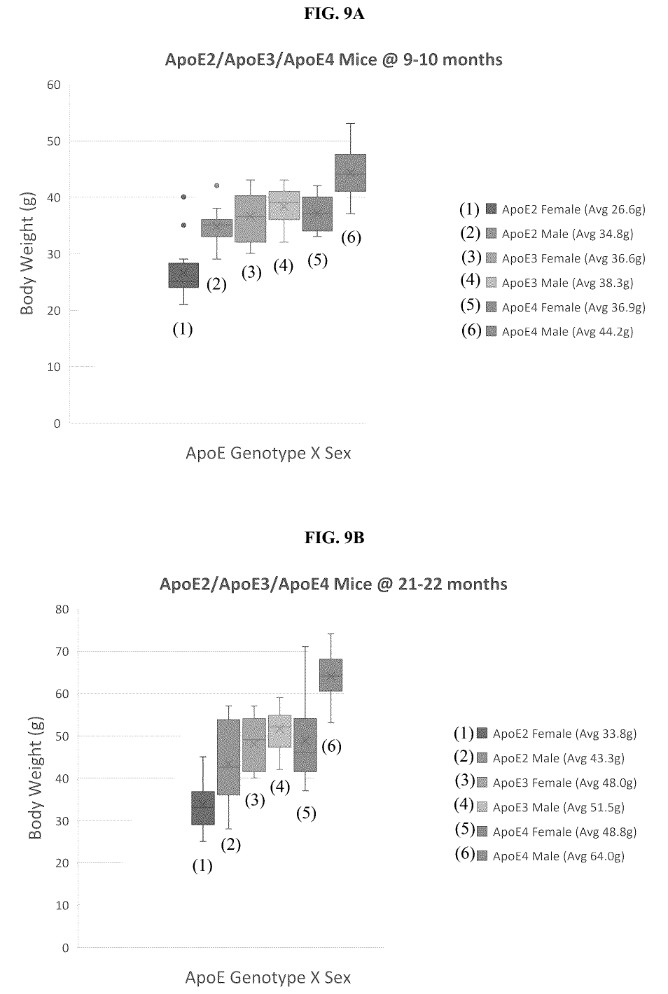

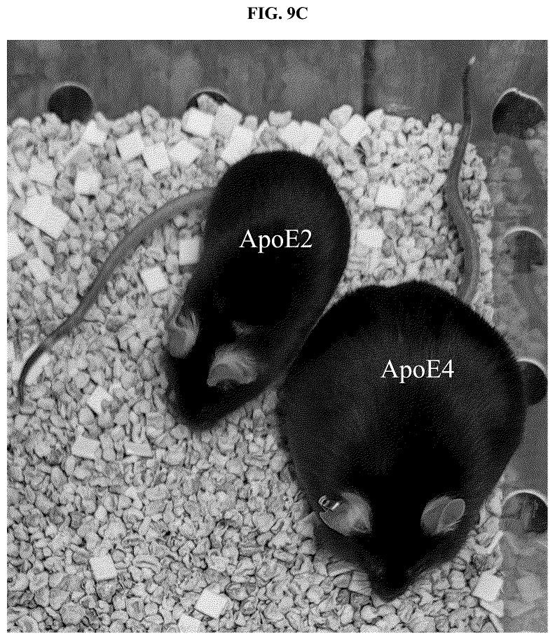

The data demonstrate the role of rhApoE2 in ameliorating AD-associated DHA dysregulation. These results indicate that a rhApoE2-based therapy not only is unlikely to induce hyperlipidemia but also has the promise to reshape the circulating lipidome, resulting in a reduced risk for the development of metabolic syndrome such as diabetes, obesity (FIGS. 9A-9B), cardiovascular disease, and AD.

TABLE 4
Lipids significantly altered by rhApoE2 in male mice serum.

| | | | | | | Log10(p- | |
|---|---|---|---|---|---|---|---|
| Lipid Name | AVE(G1) | AVE(G2) | FC(G1/G2) | Log2(FC) | T-Test | value) | VIP |
| Cer(d12:0_18:1) | 7.5133 | 22.5884 | 0.3326 | −1.5881 | 0.0185 | 1.7319 | 1.6716 |
| Cer(d16:0_16:1) | 61.9669 | 171.2588 | 0.3618 | −1.4666 | 0.0050 | 2.3005 | 1.8643 |
| Cer(d14:0_18:2) | 14.7296 | 36.9732 | 0.3984 | −1.3278 | 0.0164 | 1.7860 | 1.6933 |
| Cer(d16:0_18:1) | 243.9449 | 620.0543 | 0.3934 | −1.3458 | 0.0151 | 1.8223 | 1.7075 |
| Cer(d18:1_16:0 + O) | 21.2853 | 65.9508 | 0.3227 | −1.6315 | 0.0018 | 2.7568 | 1.9743 |
| Cer(d18:1_16:1) | 298.9723 | 716.5103 | 0.4173 | −1.2610 | 0.0266 | 1.5745 | 1.6033 |
| Cer(d16:0_18:3) | 19.1851 | 58.7901 | 0.3263 | −1.6156 | 0.0047 | 2.3312 | 1.8728 |
| Cer(d18:0_18:1) | 45.2936 | 99.7139 | 0.4542 | −1.1385 | 0.0212 | 1.6729 | 1.6469 |
| Cer(d16:0_20:1 + O) | 17.9673 | 38.9402 | 0.4614 | −1.1159 | 0.0040 | 2.3947 | 1.8898 |
| Cer(d18:1_18:0 + O) | 94.5367 | 261.1969 | 0.3619 | −1.4662 | 0.0052 | 2.2823 | 1.8593 |
| Cer(d18:0_18:2) | 39.1658 | 83.7741 | 0.4675 | −1.0969 | 0.0378 | 1.4223 | 1.5294 |
| Cer(d15:2_21:0 + O) | 16.1609 | 32.6996 | 0.4942 | −1.0168 | 0.0105 | 1.9775 | 1.7641 |
| Cer(d18:1_18:1 + O) | 71.3084 | 193.7870 | 0.3680 | −1.4423 | 0.0040 | 2.4012 | 1.8915 |
| Cer(d16:2_20:1 + O) | 7.1749 | 21.9272 | 0.3272 | −1.6117 | 0.0024 | 2.6128 | 1.9431 |
| Cer(d20:2_18:2 + 2O) | 11.8491 | 33.0816 | 0.3582 | −1.4812 | 0.0016 | 2.7987 | 1.9829 |
| Cer(d40:2) | 13.7269 | 30.3504 | 0.4523 | −1.1447 | 0.0168 | 1.7747 | 1.6888 |
| Cer(m18:1_18:1 + O) | 38.2053 | 82.9574 | 0.4605 | −1.1186 | 0.0346 | 1.4610 | 1.5491 |
| Cer(t18:0_16:1) | 21.2891 | 67.0192 | 0.3177 | −1.6545 | 0.0016 | 2.7934 | 1.9818 |
| Cer(t18:0_18:1) | 94.5367 | 261.1969 | 0.3619 | −1.4662 | 0.0052 | 2.2823 | 1.8593 |
| Cer(t18:0_18:2) | 66.8702 | 203.0090 | 0.3294 | −1.6021 | 0.0037 | 2.4279 | 1.8984 |
| Cer(t16:0_20:4) | 30.5350 | 86.8599 | 0.3515 | −1.5082 | 0.0053 | 2.2738 | 1.8569 |
| ChE(18:1) | 27.5904 | 107.0391 | 0.2578 | −1.9559 | 0.0013 | 2.8812 | 1.9992 |
| ChE(18:2) | 6265.8251 | 2342.8142 | 2.6745 | 1.4193 | 0.0367 | 1.4348 | 1.5359 |
| ChE(22:6) | 16.8377 | 40.5418 | 0.4153 | −1.2677 | 0.0311 | 1.5073 | 1.5719 |
| CmE(17:1) | 33.7964 | 107.0391 | 0.3157 | −1.6632 | 0.0011 | 2.9530 | 2.0126 |
| DG(4:0_14:0) | 7.2610 | 16.9209 | 0.4291 | −1.2206 | 0.0017 | 2.7590 | 1.9747 |
| DG(28:1e) | 1.1625 | 3.4769 | 0.3344 | −1.5805 | 0.0345 | 1.4622 | 1.5495 |
| DG(29:1e) | 44.1814 | 136.3330 | 0.3241 | −1.6256 | 0.0348 | 1.4584 | 1.5479 |
| DG(34:1e) | 11.3847 | 26.2693 | 0.4334 | −1.2063 | 0.0353 | 1.4527 | 1.5449 |
| DG(17:0_18:1) | 10.6044 | 28.0088 | 0.3786 | −1.4012 | 0.0036 | 2.4454 | 1.9029 |
| DG(17:0_18:2) | 9.2498 | 25.3110 | 0.3654 | −1.4523 | 0.0033 | 2.4764 | 1.9106 |
| DG(39:0) | 2764.7898 | 1024.8556 | 2.6977 | 1.4317 | 0.0375 | 1.4260 | 1.5314 |
| MG(28:1) | 2.7427 | 1.1811 | 2.3221 | 1.2154 | 0.0118 | 1.9284 | 1.7469 |
| MG(29:1) | 42.5318 | 136.3330 | 0.3120 | −1.6805 | 0.0338 | 1.4710 | 1.5541 |
| MG(30:1) | 0.2253 | 0.6118 | 0.3683 | −1.4412 | 0.0406 | 1.3911 | 1.5134 |
| MG(34:1) | 6.1399 | 25.0871 | 0.2447 | −2.0307 | 0.0351 | 1.4544 | 1.5459 |
| MePC(37:2) | 885.5643 | 395.0759 | 2.2415 | 1.1645 | 0.0363 | 1.4395 | 1.5382 |
| PC(18:2_20:4) | 1413.8210 | 586.1889 | 2.4119 | 1.2702 | 0.0234 | 1.6307 | 1.6285 |
| PC(40:5) | 771.0323 | 269.7062 | 2.8588 | 1.5154 | 0.0131 | 1.8820 | 1.7299 |
| SIE(16:1) | 34.0231 | 107.0391 | 0.3179 | −1.6536 | 0.0011 | 2.9575 | 2.0135 |
| TG(12:0e_8:0_19:0) | 2764.7898 | 1024.8556 | 2.6977 | 1.4317 | 0.0375 | 1.4260 | 1.5314 |
| TG(24:1_10:3_10:4) | 12.7959 | 32.5134 | 0.3936 | −1.3454 | 0.0263 | 1.5800 | 1.6059 |
| TG(16:0_16:0_18:2) | 43.9808 | 422.6034 | 0.1041 | −3.2644 | 0.0153 | 1.8151 | 1.7046 |
| TG(27:0_6:0_19:1) | 430.1459 | 874.2716 | 0.4920 | −1.0233 | 0.0381 | 1.4196 | 1.5281 |
| TG(18:0_16:1_18:1) | 137.0329 | 334.5720 | 0.4096 | −1.2878 | 0.0120 | 1.9215 | 1.7442 |
| TG(28:0_10:0_14:3) | 34.1651 | 222.6564 | 0.1534 | −2.7042 | 0.0100 | 1.9986 | 1.7711 |
| TG(28:1_10:4_18:2) | 67.4547 | 5.5386 | 12.1791 | 3.6063 | 0.0048 | 2.3210 | 1.8701 |
| TG(18:2_18:2_22:6) | 201.6482 | 98.7118 | 2.0428 | 1.0305 | 0.0091 | 2.0408 | 1.7855 |
| TG(18:3_18:2_22:6) | 57.3991 | 26.8401 | 2.1386 | 1.0966 | 0.0167 | 1.7761 | 1.6895 |
| TG(58:9) | 11.9094 | 4.9514 | 2.4053 | 1.2662 | 0.0429 | 1.3675 | 1.5007 |
| TG(20:4_20:4_20:4) | 107.6040 | 32.7955 | 3.2811 | 1.7142 | 0.0053 | 2.2748 | 1.8572 |
| TG(20:5_18:2_22:6) | 19.3560 | 2.1297 | 9.0888 | 3.1841 | 0.0145 | 1.8399 | 1.7142 |
| TG(62:13) | 9.0186 | 2.0629 | 4.3717 | 2.1282 | 0.0054 | 2.2672 | 1.8550 |
| TG(18:2_22:6_22:6) | 39.5149 | 7.0535 | 5.6022 | 2.4860 | 0.0104 | 1.9840 | 1.7663 |
| WE(2:0_14:2) | 5.1764 | 18.3991 | 0.2813 | −1.8296 | 0.0023 | 2.6398 | 1.9491 |
| WE(5:0_12:0) | 1.4409 | 378.0766 | 0.0038 | −8.0356 | 0.0042 | 2.3747 | 1.8844 |
| WE(3:0_14:0) | 1.1096 | 288.6014 | 0.0038 | −8.0228 | 0.0410 | 1.3869 | 1.5110 |
| WE(5:0_14:2) | 0.7033 | 1.7928 | 0.3923 | −1.3499 | 0.0186 | 1.7312 | 1.6711 |
| WE(16:0_18:1) | 5.8336 | 12.2690 | 0.4755 | −1.0726 | 0.0037 | 2.4284 | 1.8985 |
| ZyE(18:1) | 6267.2688 | 2342.5431 | 2.6754 | 1.4198 | 0.0367 | 1.4354 | 1.5362 |
| ZyE(20:2) | 200.8478 | 14.5994 | 13.7573 | 3.7821 | 0.0315 | 1.5014 | 1.5689 |

G1: rhApoE2-treated group;
G2: vehicle alone-treated control group

TABLE 5
Lipids significantly altered by rhApoE2 in male mice serum.

| | | | | | | Log10(p- | |
|---|---|---|---|---|---|---|---|
| Lipid Name | AVE(G3) | AVE(G4) | FC(G3/G4) | Log2(FC) | T-Test | value) | VIP |
| ChE(18:1) | 59.7423 | 10.1222 | 5.9021 | 2.5612 | 0.0013 | 2.9021 | 2.6683 |
| ChE(18:2) | 124.4714 | 41.0741 | 3.0304 | 1.5995 | 0.0023 | 2.6297 | 2.5908 |
| CmE(17:1) | 59.7423 | 5.4398 | 10.9825 | 3.4571 | 0.0002 | 3.7818 | 2.8385 |
| DG(16:0e) | 5.7587 | 2.7779 | 2.0730 | 1.0518 | 0.0187 | 1.7286 | 2.2239 |
| DG(16:1e) | 0.6020 | 0.2516 | 2.3927 | 1.2587 | 0.0348 | 1.4586 | 2.0582 |
| DG(28:1e) | 8.4182 | 1.7572 | 4.7906 | 2.2602 | 0.0497 | 1.3038 | 1.9499 |
| DG(29:1e) | 2.9217 | 9.1911 | 0.3179 | −1.6534 | 0.0273 | 1.5641 | 2.1250 |
| MG(28:1) | 3.4580 | 1.3890 | 2.4897 | 1.3160 | 0.0397 | 1.4016 | 2.0208 |
| MG(29:1) | 12.3586 | 4.0588 | 3.0448 | 1.6064 | 0.0272 | 1.5655 | 2.1290 |
| MePC(35:2) | 12.9646 | 4.5282 | 2.8631 | 1.5176 | 0.0346 | 1.4608 | 2.0605 |
| PC(8:1e_12:3) | 92.8708 | 41.4550 | 2.2403 | 1.1637 | 0.0150 | 1.8226 | 2.2695 |
| PE(16:0e) | 2.3870 | 6.3935 | 0.3734 | −1.4214 | 0.0036 | 2.4433 | 2.5342 |
| PE(12:0e_6:0) | 2.4237 | 6.5576 | 0.3696 | −1.4360 | 0.0033 | 2.4756 | 2.5443 |
| PE(18:1e) | 1.4112 | 3.4536 | 0.4086 | −1.2912 | 0.0212 | 1.6739 | 2.1952 |
| PE(20:3e) | 0.8291 | 2.2171 | 0.3740 | −1.4191 | 0.0364 | 1.4395 | 2.0484 |
| PE(18:0_20:4) | 1.9418 | 5.5101 | 0.3524 | −1.5047 | 0.0223 | 1.6518 | 2.1811 |
| PE(19:0_20:5) | 781.5325 | 354.3285 | 2.2057 | 1.1412 | 0.0489 | 1.3107 | 1.9519 |
| SiE(16:1) | 59.7423 | 6.5215 | 9.1608 | 3.1955 | 0.0002 | 3.7464 | 2.8333 |
| TG(16:0_16:0_18:2) | 274.1060 | 118.1182 | 2.3206 | 1.2145 | 0.0164 | 1.7849 | 2.2552 |
| TG(16:1_18:2_18:2) | 860.8602 | 134.4948 | 6.4007 | 2.6782 | 0.0406 | 1.3920 | 2.0151 |
| TG(16:0_16:1_20:4) | 41.2302 | 10.0780 | 4.0911 | 2.0325 | 0.0200 | 1.6993 | 2.2035 |
| TG(18:1_18:1_18:2) | 49.6577 | 15.6051 | 3.1821 | 1.6700 | 0.0313 | 1.5051 | 2.0902 |
| TG(14:1_21:1_21:1) | 969.0399 | 211.5381 | 4.5809 | 2.1956 | 0.0268 | 1.5717 | 2.1316 |
| TG(18:1_18:1_20:3) | 288.1163 | 56.7483 | 5.0771 | 2.3440 | 0.0294 | 1.5324 | 2.1067 |
| TG(18:2_18:2_20:4) | 280.8424 | 74.7498 | 3.7571 | 1.9096 | 0.0190 | 1.7219 | 2.2215 |
| TG(18:1_18:2_22:5) | 57.9277 | 23.1714 | 2.5000 | 1.3219 | 0.0179 | 1.7467 | 2.2335 |
| WE(5:0_14:2) | 0.9369 | 1.9132 | 0.4897 | −1.0299 | 0.0478 | 1.3204 | 1.9641 |
| ZyE(18:0) | 54.5538 | 5.4886 | 9.9395 | 3.3132 | 0.0087 | 2.0613 | 2.3889 |
| ZyE(22:5) | 5.6912 | 2.1015 | 2.7082 | 1.4373 | 0.0306 | 1.5139 | 2.0967 |

G1: rhApoE2-treated group;
G2: vehicle alone-treated control group

The disparities in body weight between ApoE2 and ApoE4 KI mice reflect severe lipid dyshomeostasis associated with ApoE4 (FIG. 9C), which will be further confirmed by the lipidomics data. Moreover, the lipidomics data will confirm the classes of lipids are dysregulated in ApoE4 mice.

### Example 9: Effects of rhApoE2 Treatment in Obesity Model

Recent research has indicated that elevated levels of ceramides in the circulation play a central mechanistic role in developing insulin resistance and metabolic disorders such as fatty liver disease, type 2 diabetes, and cardiovascular disease. Obesity increases the risk for insulin resistance and metabolism syndrome, and has also been associated with increased circulating levels of ceramides. By lowering circulating ceramides, rhApoE2 treatment is expected to reverse insulin resistance and, consequently, attenuate the development and progression of metabolic syndrome.

We plan to run a two-month rhApoE2 treatment study in two different mouse models of obesity. A genetic model of obesity: B6.Cg-Lep^{ob}/J (B6 ob)—will be obtained from JAX Labs (000632) and a life-style-related model of obesity: C57BL/6J Diet-Induced Obesity (B6 DIO)—will be obtained from JAX Lab (380050) rhApoE2 treatment will be initiated when mice reach 12 weeks of age. Mice will be treated by intravenous administration of rhApoE2 vial tail vein injection, once weekly, for eight weeks. At the end of the two-month treatment, mice will be subjected to glucose and insulin tolerance tests. Mice will then be euthanized, and blood, liver, spleen, heart, and brain tissue will be collected for biochemical and histological analyses. The blood will be processed into serum samples for metabolic and lipid profile analyses. We also plan to run a two-month treatment study in mid-aged ApoE4 mice (15-16 weeks) with the same design.

It is anticipated that rhApoE2 treatment will result in one or more of the following positive outcomes in both models: (a) rhApoE2 treatment will improve the overall circulating metabolic and lipidome profile, including decreased levels of ceramides, (b) rhApoE2 treatment will improve glucose tolerance and insulin resistance, with increased insulin signaling activity, (c) rhApoE2 treatment will attenuate proinflammatory profiles as indicated by decreased levels of cytokines in the blood and tissues, (d) rhApoE2 treatment will attenuate hepatic steatosis (fatty liver), (e) rhApoE2 treatment will induce some positive changes in the brain, and (f) rhApoE2 treatment will significantly reduce the body weight of aged ApoE4 mice.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Costantini L C, Barr L J, Vogel J L, Henderson S T. Hypometabolism as a therapeutic target in Alzheimer's disease. BMC Neuroscience 2008; 9: S16-S.
2. Yamane K, Yokono K, Okada Y. Anaerobic glycolysis is crucial for the maintenance of neural activity in guinea pig hippocampal slices. Journal of neuroscience methods 2000; 103:163-71.
3. Fleck M W, Henze D A, Barrionuevo G, Palmer A M. Aspartate and glutamate mediate excitatory synaptic transmission in area CA1 of the hippocampus. The Journal of neuroscience: the official journal of the Society for Neuroscience 1993; 13:3944-55.
4. Takata T, Okada Y. Effects of deprivation of oxygen or glucose on the neural activity in the guinea pig hippocampal slice—intracellular recording study of pyramidal neurons. Brain research 1995; 683:109-16.
5. Langbaum J B, Chen K, Lee W, et al. Categorical and correlational analyses of baseline fluorodeoxyglucose positron emission tomography images from the Alzheimer's Disease Neuroimaging Initiative (ADNI). Neuroimage 2009; 45:1107-16.
6. Small G W, Mazziotta J C, Collins M T, et al. Apolipoprotein E type 4 allele and cerebral glucose metabolism in relatives at risk for familial Alzheimer disease. Jama 1995; 273:942-7.
7. Mosconi L, De Santi S, Li J, et al. Hippocampal hypometabolism predicts cognitive decline from normal aging. Neurobiology of aging 2008; 29:676-92.
8. Mosconi L, Sorbi S, de Leon M J, et al. Hypometabolism exceeds atrophy in presymptomatic early-onset familial Alzheimer's disease. Journal of nuclear medicine: official publication, Society of Nuclear Medicine 2006; 47:1778-86.
9. de Leon M J, Convit A, Wolf O T, et al. Prediction of cognitive decline in normal elderly subjects with 2-[(18)F] fluoro-2-deoxy-d-glucose/positron-emission tomography (FDG/PET). Proceedings of the National Academy of Sciences of the United States of America 2001; 98:10966-71.
10. Malonek D, Grinvald A. Interactions between electrical activity and cortical microcirculation revealed by imaging spectroscopy: implications for functional brain mapping. Science (New York, N Y) 1996; 272:551-4.
11. Attwell D, Iadecola C. The neural basis of functional brain imaging signals. Trends in neurosciences 2002; 25:621-5.
12. Rocher A B, Chapon F, Blaizot X, Baron J C, Chavoix C. Resting-state brain glucose utilization as measured by PET is directly related to regional synaptophysin levels: a study in baboons. Neuroimage 2003; 20:1894-8.
13. Khatri N, Man H-Y. Synaptic Activity and Bioenergy Homeostasis: Implications in Brain Trauma and Neurodegenerative Diseases. Frontiers in Neurology 2013; 4:199.
14. Mosconi L. Brain glucose metabolism in the early and specific diagnosis of Alzheimer's disease. FDG-PET studies in MCI and AD. European journal of nuclear medicine and molecular imaging 2005; 32:486-510.
15. Mosconi L, De Santi S, Brys M, et al. Hypometabolism and altered cerebrospinal fluid markers in normal apolipoprotein E E4 carriers with subjective memory complaints. Biological psychiatry 2008; 63:609-18.
16. Reiman E M, Caselli R J, Chen K, Alexander G E, Bandy D, Frost J. Declining brain activity in cognitively normal apolipoprotein E epsilon 4 heterozygotes: A foundation for using positron emission tomography to efficiently test treatments to prevent Alzheimer's disease. Proc Natl Acad Sci USA 2001; 98:3334-9.
17. Reiman E M, Chen K, Alexander G E, et al. Functional brain abnormalities in young adults at genetic risk for late-onset Alzheimer's dementia. Proc Natl Acad Sci USA 2004; 101:284-9.
18. Reiman E M, Caselli R J, Yun L S, et al. Preclinical evidence of Alzheimer's disease in persons homozygous for the epsilon 4 allele for apolipoprotein E. The New England journal of medicine 1996; 334:752-8.
19. Zielke H R, Zielke C L, Baab P J. Direct measurement of oxidative metabolism in the living brain by microdialysis: a review. J Neurochem 2009; 109 Suppl 1:24-9.
20. Owen O E. Ketone bodies as a fuel for the brain during starvation. Biochemistry and Molecular Biology Education 2006; 33:246-51.
21. Kam K, Nicoll R. Excitatory Synaptic Transmission Persists Independently of the Glutamate-Glutamine Cycle. The Journal of Neuroscience 2007; 27:9192.
22. Magistretti P J, Allaman I. Lactate in the brain: from metabolic end-product to signalling molecule. Nature Reviews Neuroscience 2018; 19:235-49.
23. Yellen G. Fueling thought: Management of glycolysis and oxidative phosphorylation in neuronal metabolism. The Journal of cell biology 2018; 217:2235-46.
24. Pfeiffer T, Schuster S, Bonhoeffer S. Cooperation and competition in the evolution of ATP-producing pathways. Science 2001; 292:504-7.
25. Ghesquiere B, Wong B W, Kuchnio A, Carmeliet P. Metabolism of stromal and immune cells in health and disease. Nature 2014; 511:167-76.
26. Voloboueva L A, Emery J F, Sun X, Giffard R G. Inflammatory response of microglial BV-2 cells includes a glycolytic shift and is modulated by mitochondrial glucose-regulated protein 75/mortalin. FEBS Lett 2013; 587:756-62.
27. Schmitz J P, Groenendaal W, Wessels B, et al. Combined in vivo and in silico investigations of activation of glycolysis in contracting skeletal muscle. Am J Physiol Cell Physiol 2013; 304: C180-93.
28. Pearce E L, Poffenberger M C, Chang C H, Jones R G. Fueling immunity: insights into metabolism and lymphocyte function. Science 2013; 342:1242454.
29. Pellerin L, Bouzier-Sore A K, Aubert A, et al. Activity-dependent regulation of energy metabolism by astrocytes: an update. Glia 2007; 55:1251-62.
30. Hoyer S, Nitsch R, Oesterreich K. Predominant abnormality in cerebral glucose utilization in late-onset dementia of the Alzheimer type: a cross-sectional comparison against advanced late-onset and incipient early-onset cases. Journal of neural transmission Parkinson's disease and dementia section 1991; 3:1-14.
31. Fukuyama H, Ogawa M, Yamauchi H, et al. Altered cerebral energy metabolism in Alzheimer's disease: a PET study. Journal of nuclear medicine: official publication, Society of Nuclear Medicine 1994; 35:1-6.
32. An Y, Varma V R, Varma S, et al. Evidence for brain glucose dysregulation in Alzheimer's disease. Alzheimer's & dementia: the journal of the Alzheimer's Association 2018; 14:318-29.
33. Vlassenko A G, Gordon B A, Goyal M S, et al. Aerobic glycolysis and tau deposition in preclinical Alzheimer's disease. Neurobiology of aging 2018; 67:95-8.
34. Bergau N, Maul S, Rujescu D, Simm A, Navarrete Santos A. Reduction of Glycolysis Intermediate Concentrations in the Cerebrospinal Fluid of Alzheimer's Disease Patients. Frontiers in neuroscience 2019; 13:871-.
35. Lauretti E, Li J G, Di Meco A, Praticò D. Glucose deficit triggers tau pathology and synaptic dysfunction in a tauopathy mouse model. Translational psychiatry 2017; 7: e1020-e.
36. Vosseller K, Trinidad J C, Chalkley R J, et al. O-linked N-acetylglucosamine proteomics of postsynaptic density preparations using lectin weak affinity chromatography and mass spectrometry. Molecular & cellular proteomics: MCP 2006; 5:923-34.
37. Skorobogatko Y V, Deuso J, Adolf-Bryfogle J, et al. Human Alzheimer's disease synaptic O-GlcNAc site mapping and iTRAQ expression proteomics with ion trap mass spectrometry. Amino acids 2011; 40:765-79.
38. Trinidad J C, Barkan D T, Gulledge B F, et al. Global identification and characterization of both O-GlcNAcylation and phosphorylation at the murine synapse. Molecular & cellular proteomics: MCP 2012; 11:215-29.
39. Zhang X, Bennett V. Identification of O-linked N-acetylglucosamine modification of ankyrinG isoforms targeted to nodes of Ranvier. The Journal of biological chemistry 1996; 271:31391-8.
40. Gu J H, Shi J, Dai C L, et al. O-GlcNAcylation Reduces Ischemia-Reperfusion-Induced Brain Injury. Sci Rep 2017; 7:10686.
41. Groves J A, Lee A, Yildirir G, Zachara N E. Dynamic O-GlcNAcylation and its roles in the cellular stress response and homeostasis. Cell Stress Chaperones 2013; 18:535-58.
42. Jacobsen K T, Iverfeldt K. O-GlcNAcylation increases non-amyloidogenic processing of the amyloid-β precursor protein (APP). Biochemical and biophysical research communications 2011; 404:882-6.
43. Chun Y S, Kwon O H, Chung S. O-GlcNAcylation of amyloid-β precursor protein at threonine 576 residue regulates trafficking and processing. Biochemical and biophysical research communications 2017; 490:486-91.
44. Liu F, Shi J, Tanimukai H, et al. Reduced O-GlcNAcylation links lower brain glucose metabolism and tau pathology in Alzheimer's disease. Brain: a journal of neurology 2009; 132:1820-32.
45. Achariyar T M, Li B, Peng W, et al. Glymphatic distribution of CSF-derived apoE into brain is isoform specific and suppressed during sleep deprivation. Mol Neurodegener 2016; 11:74-.
46. Uchihara T, Duyckaerts C, He Y, et al. ApoE immunoreactivity and microglial cells in Alzheimer's disease brain. Neuroscience letters 1995; 195:5-8.
47. Knoferle J, Yoon S Y, Walker D, et al. Apolipoprotein E4 produced in GABAergic interneurons causes learning and memory deficits in mice. The Journal of neuroscience: the official journal of the Society for Neuroscience 2014; 34:14069-78.
48. Xu Q, Bernardo A, Walker D, Kanegawa T, Mahley R W, Huang Y. Profile and regulation of apolipoprotein E (ApoE) expression in the CNS in mice with targeting of green fluorescent protein gene to the ApoE locus. The Journal of neuroscience: the official journal of the Society for Neuroscience 2006; 26:4985-94.
49. Xu P T, Gilbert J R, Qiu H L, et al. Specific regional transcription of apolipoprotein E in human brain neurons. Am J Pathol 1999; 154:601-11.
50. Xu P T, Schmechel D, Rothrock-Christian T, et al. Human apolipoprotein E2, E3, and E4 isoform-specific transgenic mice: human-like pattern of glial and neuronal immunoreactivity in central nervous system not observed in wild-type mice. Neurobiol Dis 1996; 3:229-45.
51. Dekroon R M, Armati P J. Synthesis and processing of apolipoprotein E in human brain cultures. Glia 2001; 33:298-305.
52. Boschert U, Merlo-Pich E, Higgins G, Roses A D, Catsicas S. Apolipoprotein E Expression by Neurons Surviving Excitotoxic Stress. Neurobiology of Disease 1999; 6:508-14.
53. Aoki K, Uchihara T, Sanjo N, et al. Increased expression of neuronal apolipoprotein E in human brain with cerebral infarction. Stroke 2003; 34:875-80.
54. Theendakara V, Peters-Libeu C A, Spilman P, Poksay K S, Bredesen D E, Rao R V. Direct Transcriptional Effects of Apolipoprotein E. The Journal of neuroscience: the official journal of the Society for Neuroscience 2016; 36:685-700.
55. Wu L, Zhang X, Zhao L. Human ApoE Isoforms Differentially Modulate Brain Glucose and Ketone Body Metabolism: Implications for Alzheimer's Disease Risk Reduction and Early Intervention. The Journal of neuroscience: the official journal of the Society for Neuroscience 2018; 38:6665-81.
56. Wu L, Zhao L. ApoE2 and Alzheimer's disease: time to take a closer look. Neural Regen Res 2016; 11:412-3.
57. Keeney J T, Ibrahimi S, Zhao L. Human ApoE Isoforms Differentially Modulate Glucose and Amyloid Metabolic Pathways in Female Brain: Evidence of the Mechanism of Neuroprotection by ApoE2 and Implications for Alzheimer's Disease Prevention and Early Intervention. J Alzheimers Dis 2015; 48:411-24.
58. Woody S K, Zhou H, Ibrahimi S, Dong Y, Zhao L. Human ApoE ε2 Promotes Regulatory Mechanisms of Bioenergetic and Synaptic Function in Female Brain: A Focus on V-type H+-ATPase. J Alzheimers Dis 2016; 53:1015-31.
59. Zhang X, Wu L, Swerdlow R H, Zhao L. Opposing Effects of ApoE2 and ApoE4 on Glycolytic Metabolism in Neuronal Aging Supports a Warburg Neuroprotective Cascade against Alzheimer's Disease. Cells 2023; 12.
60. Newington J T, Pitts A, Chien A, Arseneault R, Schubert D, Cumming R C. Amyloid beta resistance in nerve cell lines is mediated by the Warburg effect. PloS one 2011; 6: e19191.
61. Newington J T, Rappon T, Albers S, Wong D Y, Rylett R J, Cumming R C. Overexpression of pyruvate dehydrogenase kinase 1 and lactate dehydrogenase A in nerve cells confers resistance to amyloid beta and other toxins by decreasing mitochondrial respiration and reactive oxygen species production. The Journal of biological chemistry 2012; 287:37245-58.
62. Turpin-Nolan S M, Bruning J C. The role of ceramides in metabolic disorders: when size and localization matters. Nat Rev Endocrinol 2020; 16:224-33.
63. Summers S A, Chaurasia B, Holland W L. Metabolic Messengers: ceramides. Nat Metab 2019; 1:1051-8.
64. Chavez J A, Summers S A. A ceramide-centric view of insulin resistance. Cell Metab 2012; 15:585-94.
65. Sokolowska E, Blachnio-Zabielska A. The Role of Ceramides in Insulin Resistance. Front Endocrinol (Lausanne) 2019; 10:577.
66. Tippetts T S, Holland W L, Summers S A. Cholesterol—the devil you know; ceramide—the devil you don't. Trends Pharmacol Sci 2021; 42:1082-95.
67. Meeusen J W, Donato L J, Bryant S C, Baudhuin L M, Berger P B, Jaffe A S. Plasma Ceramides. Arterioscler Thromb Vasc Biol 2018; 38:1933-9.
68. Hilvo M, Meikle P J, Pedersen E R, et al. Development and validation of a ceramide- and phospholipid-based cardiovascular risk estimation score for coronary artery disease patients. Eur Heart J 2020; 41:371-80.
69. Mielke M M, Bandaru V V, Haughey N J, Rabins P V, Lyketsos C G, Carlson M C. Serum sphingomyelins and ceramides are early predictors of memory impairment. Neurobiol Aging 2010; 31:17-24.
70. Mielke M M, Bandaru V V, Haughey N J, et al. Serum ceramides increase the risk of Alzheimer disease: the Women's Health and Aging Study I I. Neurology 2012; 79:633-41.
71. Mielke M M, Haughey N J, Bandaru V V, et al. Plasma ceramides are altered in mild cognitive impairment and predict cognitive decline and hippocampal volume loss. Alzheimers Dement 2010; 6:378-85.
72. Mielke M M, Haughey N J, Han D, et al. The Association Between Plasma Ceramides and Sphingomyelins and Risk of Alzheimer's Disease Differs by Sex and APOE in the Baltimore Longitudinal Study of Aging. J Alzheimers Dis 2017; 60:819-28.
73. Xia J Y, Holland W L, Kusminski C M, et al. Targeted Induction of Ceramide Degradation Leads to Improved Systemic Metabolism and Reduced Hepatic Steatosis. Cell Metab 2015; 22:266-78.
74. Chaurasia B, Tippetts T S, Mayoral Monibas R, et al. Targeting a ceramide double bond improves insulin resistance and hepatic steatosis. Science 2019; 365:386-92.
75. Kusminski C M, Scherer P E. Lowering ceramides to overcome diabetes. Science 2019; 365:319-20.
76. Summers S A. The ART of Lowering Ceramides. Cell Metab 2015; 22:195-6. 77 Piccolis M, Bond L M, Kampmann M, et al. Probing the Global Cellular Responses to Lipotoxicity Caused by Saturated Fatty Acids. Mol Cell 2019; 74:32-44 e8.
78. Tove S B. Toxicity of Saturated Fat. J Nutr 1964; 84:237-43.
79 Listenberger L L, Han X, Lewis S E, et al. Triglyceride accumulation protects against fatty acid-induced lipotoxicity. Proc Natl Acad Sci USA 2003; 100:3077-82.
80. Gehrmann W, Wurdemann W, Plotz T, Jorns A, Lenzen S, Elsner M. Antagonism Between Saturated and Unsaturated Fatty Acids in ROS Mediated Lipotoxicity in Rat Insulin-Producing Cells. Cell Physiol Biochem 2015; 36:852-65.
81. Thombare K, Ntika S, Wang X, Krizhanovskii C. Long chain saturated and unsaturated fatty acids exert opposing effects on viability and function of GLP-1-producing cells: Mechanisms of lipotoxicity. PLOS One 2017; 12: e0177605.
82. Plotz T, Krummel B, Laporte A, et al. The monounsaturated fatty acid oleate is the major physiological toxic free fatty acid for human beta cells. Nutr Diabetes 2017; 7:305.
83. Plotz T, von Hanstein A S, Krummel B, Laporte A, Mehmeti I, Lenzen S. Structure-toxicity relationships of saturated and unsaturated free fatty acids for elucidating the lipotoxic effects in human EndoC-betaH1 beta-cells. Biochim Biophys Acta Mol Basis Dis 2019; 1865:165525.
84. von Hanstein A S, Lenzen S, Plotz T. Toxicity of fatty acid profiles of popular edible oils in human EndoC-betaH1 beta-cells. Nutr Diabetes 2020; 10:5.
85. Fanning S, Haque A, Imberdis T, et al. Lipidomic Analysis of alpha-Synuclein Neurotoxicity Identifies Stearoyl CoA Desaturase as a Target for Parkinson Treatment. Mol Cell 2019; 73:1001-14 e8.
86. Vincent B M, Tardiff D F, Piotrowski J S, et al. Inhibiting Stearoyl-CoA Desaturase Ameliorates alpha-Synuclein Cytotoxicity. Cell Rep 2018; 25:2742-54 e31.
87. Swanson D, Block R, Mousa S A. Omega-3 fatty acids EPA and DHA: health benefits throughout life. Adv Nutr 2012; 3:1-7.
88. Rodriguez D, Lavie C J, Elagizi A, Milani R V. Update on Omega-3 Polyunsaturated Fatty Acids on Cardiovascular Health. Nutrients 2022; 14.
89. Kitajka K, Sinclair A J, Weisinger R S, et al. Effects of dietary omega-3 polyunsaturated fatty acids on brain gene expression. Proc Natl Acad Sci USA 2004; 101:10931-6.
90. Sinclair A J. Long-chain polyunsaturated fatty acids in the mammalian brain. Proc Nutr Soc 1975; 34:287-91.
91. Bazinet R P, Laye S. Polyunsaturated fatty acids and their metabolites in brain function and disease. Nat Rev Neurosci 2014; 15:771-85.
92. Martinat M, Rossitto M, Di Miceli M, Laye S. Perinatal Dietary Polyunsaturated Fatty Acids in Brain Development, Role in Neurodevelopmental Disorders. Nutrients 2021; 13.
93. Innis S M. Dietary (n-3) fatty acids and brain development. J Nutr 2007; 137:855-9.
94. Howe P R C, Evans H M, Kuszewski J C, Wong R H X. Effects of Long Chain Omega-3 Polyunsaturated Fatty Acids on Brain Function in Mildly Hypertensive Older Adults. Nutrients 2018; 10.
95. Jerneren F, Elshorbagy A K, Oulhaj A, Smith S M, Refsum H, Smith A D. Brain atrophy in cognitively impaired elderly: the importance of long-chain omega-3 fatty acids and B vitamin status in a randomized controlled trial. Am J Clin Nutr 2015; 102:215-21.
96. Witte A V, Kerti L, Hermannstadter H M, et al. Long-chain omega-3 fatty acids improve brain function and structure in older adults. Cereb Cortex 2014; 24:3059-68.
97. Zamroziewicz M K, Paul E J, Zwilling C E, Barbey A K. Determinants of fluid intelligence in healthy aging: Omega-3 polyunsaturated fatty acid status and frontoparietal cortex structure. Nutr Neurosci 2018; 21:570-9.
98. Gu Y, Vorburger R S, Gazes Y, et al. White matter integrity as a mediator in the relationship between dietary nutrients and cognition in the elderly. Ann Neurol 2016; 79:1014-25.
99. Tan Z S, Harris W S, Beiser A S, et al. Red blood cell omega-3 fatty acid levels and markers of accelerated brain aging. Neurology 2012; 78:658-64.
100. Bernath M M, Bhattacharyya S, Nho K, et al. Serum triglycerides in Alzheimer disease: Relation to neuroimaging and CSF biomarkers. Neurology 2020; 94: e2088-e98.
101. Tsukamoto K, Maugeais C, Glick J M, Rader D J. Markedly increased secretion of VLDL triglycerides induced by gene transfer of apolipoprotein E isoforms in apoE-deficient mice. J Lipid Res 2000; 41:253-9.
102. Tacito L H B, Yamada L N, de Souza Pinhel M A, Yugar-Toledo J C, Souza D R S. Influence of Apolipoprotein E on the Lipid Profile and Postprandial Triglyceride Levels in Brazilian Postmenopausal Women With Artery Disease. Clin Med Insights Cardiol 2017; 11:1179546817731110.
103. Shaw J T, Tate J, Kesting J B, et al. Apolipoprotein E polymorphism in indigenous Australians: allelic frequencies and relationship with dyslipidemia. Med J Aust 1999; 170:161-4. 104. Kuo C L, Pilling L C, Atkins J L, Kuchel G A, Melzer D. ApoE e2 and aging-related outcomes in 379,000 U K Biobank participants. Aging (Albany N Y) 2020; 12:12222-33.
105. Whitacre B E, Howles P, Street S, Morris J, Swertfeger D, Davidson W S. Apolipoprotein E content of VLDL limits LPL-mediated triglyceride hydrolysis. J Lipid Res 2022; 63:100157.
106. Gonzalez-Granillo M, Helguero L A, Alves E, et al. Sex-specific lipid molecular signatures in obesity-associated metabolic dysfunctions revealed by lipidomic characterization in ob/ob mouse. Biol Sex Differ 2019; 10:11.
107. Hasty A H, Linton M F, Swift L L, Fazio S. Determination of the lower threshold of apolipoprotein E resulting in remnant lipoprotein clearance. Journal of lipid research 1999; 40:1529-38.
108. Sajesh B V, On N H, Omar R, et al. Validation of Cadherin HAV6 Peptide in the Transient Modulation of the Blood-Brain Barrier for the Treatment of Brain Tumors. Pharmaceutics 2019; 11.
109. Ulapane K R, On N, Kiptoo P, Williams T D, Miller D W, Siahaan T J. Improving Brain Delivery of Biomolecules via BBB Modulation in Mouse and Rat: Detection using MRI, NIRF, and Mass Spectrometry. Nanotheranostics 2017; 1:217-31.
110. Ulapane K R, Kopec B M, Siahaan T J. Improving In Vivo Brain Delivery of Monoclonal Antibody Using Novel Cyclic Peptides. Pharmaceutics 2019; 11.
111. Alaofi A, On N, Kiptoo P, Williams T D, Miller D W, Siahaan T J. Comparison of Linear and Cyclic His-Ala-Val Peptides in Modulating the Blood-Brain Barrier Permeability: Impact on Delivery of Molecules to the Brain. Journal of pharmaceutical sciences 2016; 105:797-807.
112. Kopec B M, Kiptoo P, Zhao L, Rosa-Molinar E, Siahaan T J. Noninvasive Brain Delivery and Efficacy of BDNF to Stimulate Neuroregeneration and Suppression of Disease Relapse in EAE Mice. Molecular pharmaceutics 2019.
113. Laksitorini M D, Kiptoo P K, On N H, Thliveris J A, Miller D W, Siahaan T J. Modulation of intercellular junctions by cyclic-ADT peptides as a method to reversibly increase blood-brain barrier permeability. Journal of pharmaceutical sciences 2015; 104:1065-75.
114. Kaneva A M, Bojko E R, Potolitsyna N N, Odland J O. Plasma levels of apolipoprotein-E in residents of the European North of Russia. Lipids in Health and Disease 2013; 12:43.
115. Mahley R W, Innerarity T L, Rall S C, Jr., Weisgraber K H. Plasma lipoproteins: apolipoprotein structure and function. Journal of lipid research 1984; 25:1277-94.
116. Mielke M M, Bandaru V V, Haughey N J, Rabins P V, Lyketsos C G, Carlson M C. Serum sphingomyelins and ceramides are early predictors of memory impairment. Neurobiol Aging 2010; 31:17-24.
117. Mielke M M, Bandaru V V, Haughey N J, et al. Serum ceramides increase the risk of Alzheimer disease: the Women's Health and Aging Study I I. Neurology 2012; 79:633-41.
118. Mielke M M, Haughey N J, Bandaru V V, et al. Plasma ceramides are altered in mild cognitive impairment and predict cognitive decline and hippocampal volume loss. Alzheimers Dement 2010; 6:378-85.
119. Mielke M M, Haughey N J, Han D, et al. The Association Between Plasma Ceramides and Sphingomyelins and Risk of Alzheimer's Disease Differs by Sex and APOE in the Baltimore Longitudinal Study of Aging. J Alzheimers Dis 2017; 60:819-28.
120. Xia J Y, Holland W L, Kusminski C M, et al. Targeted Induction of Ceramide Degradation Leads to Improved Systemic Metabolism and Reduced Hepatic Steatosis. Cell Metab 2015; 22:266-78.
121. Chaurasia B, Tippetts T S, Mayoral Monibas R, et al. Targeting a ceramide double bond improves insulin resistance and hepatic steatosis. Science 2019; 365:386-92.
122. Kusminski C M, Scherer P E. Lowering ceramides to overcome diabetes. Science 2019; 365:319-20.
123. Summers S A. The ART of Lowering Ceramides. Cell Metab 2015; 22:195-6.
124. Swanson D, Block R, Mousa S A. Omega-3 fatty acids EPA and DHA: health benefits throughout life. Adv Nutr 2012; 3:1-7.
125. Rodriguez D, Lavie C J, Elagizi A, Milani R V. Update on Omega-3 Polyunsaturated Fatty Acids on Cardiovascular Health. Nutrients 2022; 14.
126. Kitajka K, Sinclair A J, Weisinger R S, et al. Effects of dietary omega-3 polyunsaturated fatty acids on brain gene expression. Proc Natl Acad Sci USA 2004; 101:10931-6.
127. Sinclair A J. Long-chain polyunsaturated fatty acids in the mammalian brain. Proc Nutr Soc 1975; 34:287-91.
128. Bazinet R P, Laye S. Polyunsaturated fatty acids and their metabolites in brain function and disease. Nat Rev Neurosci 2014; 15:771-85.
129. Martinat M, Rossitto M, Di Miceli M, Laye S. Perinatal Dietary Polyunsaturated Fatty Acids in Brain Development, Role in Neurodevelopmental Disorders. Nutrients 2021; 13.
130. Innis S M. Dietary (n-3) fatty acids and brain development. J Nutr 2007; 137:855-9.
131. Howe P R C, Evans H M, Kuszewski J C, Wong R H X. Effects of Long Chain Omega-3 Polyunsaturated Fatty Acids on Brain Function in Mildly Hypertensive Older Adults. Nutrients 2018; 10.
132. Jerneren F, Elshorbagy A K, Oulhaj A, Smith S M, Refsum H, Smith A D. Brain atrophy in cognitively impaired elderly: the importance of long-chain omega-3 fatty acids and B vitamin status in a randomized controlled trial. Am J Clin Nutr 2015; 102:215-21.
133. Witte A V, Kerti L, Hermannstadter H M, et al. Long-chain omega-3 fatty acids improve brain function and structure in older adults. Cereb Cortex 2014; 24:3059-68.
134. Zamroziewicz M K, Paul E J, Zwilling C E, Barbey A K. Determinants of fluid intelligence in healthy aging: Omega-3 polyunsaturated fatty acid status and frontoparietal cortex structure. Nutr Neurosci 2018; 21:570-9.
135. Gu Y, Vorburger R S, Gazes Y, et al. White matter integrity as a mediator in the relationship between dietary nutrients and cognition in the elderly. Ann Neurol 2016; 79:1014-25.
136. Tan Z S, Harris W S, Beiser A S, et al. Red blood cell omega-3 fatty acid levels and markers of accelerated brain aging. Neurology 2012; 78:658-64.
137. Shang J, Yamashita T, Fukui Y, et al. Different Associations of Plasma Biomarkers in Alzheimer's Disease, Mild Cognitive Impairment, Vascular Dementia, and Ischemic Stroke. J Clin Neurol 2018; 14:29-34.
138. Bernath M M, Bhattacharyya S, Nho K, et al. Serum triglycerides in Alzheimer disease: Relation to neuroimaging and CSF biomarkers. Neurology 2020; 94: e2088-e98.
139. Zhang X, Alshakhshir N, Zhao L. Glycolytic metabolism, brain resilience, and Alzheimer's Disease. Front Neurosci 2021; 15:662242.
140. Mahley R W. Apolipoprotein E: from cardiovascular disease to neurodegenerative disorders. J Mol Med (Berl) 2016; 94:739-46.
141. Mahley R W, Huang Y, Rall S C, Jr. Pathogenesis of type III hyperlipoproteinemia (dysbetalipoproteinemia). Questions, quandaries, and paradoxes. J Lipid Res 1999; 40:1933-49.
142. Gerritsen G, van der Hoogt C C, Schaap F G, et al. ApoE2-associated hypertriglyceridemia is ameliorated by increased levels of apoA-V but unaffected by apoC-III deficiency. J Lipid Res 2008; 49:1048-55.
143. Schaefer J R, Sattler A M, Hackler B, et al. Hyperlipidemia in patients with apolipoprotein E 2/2 phenotype: apolipoprotein A5 S19W mutation as a cofactor. Clinical chemistry 2004; 50:2214.

1. A method for treating or preventing a neurodegenerative disease in a subject in need thereof comprising:
administering to the subject an effective amount of a plurality of recombinant ApoE2 (rhApoE2) polypeptides comprising SEQ ID NO: 1,
wherein the plurality of recombinant rhApoE2 polypeptides comprise O-linked glycosylation and sialylation modifications and have a molecular weight that is greater than 34 kDa.

2. The method of claim 1, wherein the neurodegenerative disease is characterized by an altered neuronal metabolism compared to that observed in a healthy control subject.

3. The method of claim 2, wherein the neuronal metabolism comprises neuronal glycolysis.

4. The method of claim 1, wherein the neurogenerative disease is Alzheimer's Disease, Parkinson's disease, Huntington's disease, or amyotrophic lateral sclerosis.

5. The method of claim 1, wherein the plurality of recombinant rhApoE2 polypeptides have molecular weights that range from 34.05 kDa to 37 kDa.

6. The method of claim 1, further comprising:
administering to the subject an effective amount of a blood-brain barrier (BBB) modulator peptide,
optionally wherein the BBB modulator peptide is an E-cadherin-derived peptide.

7. The method of claim 6, wherein the E-cadherin-derived peptide is ADTC5 or HAVN1.

8. The method of claim 1, wherein the rhApoE2 polypeptides and/or BBB modulator peptides is administered intravenously or intranasally.

9. The method of claim 1, wherein the subject comprises an ApoE genotype selected from among ApoE ε3/ε3, ApoE ε2/ε4, ApoE ε3/ε4 and ApoE ε4/ε4.

10. A method for preventing or treating a metabolic disease in a subject in need thereof comprising
administering to the subject an effective amount of a plurality of recombinant ApoE2 (rhApoE2) polypeptides comprising SEQ ID NO: 1,
wherein the plurality of recombinant rhApoE2 polypeptides comprise O-linked glycosylation and sialylation modifications and have a molecular weight that is greater than 34 kDa, optionally wherein the subject comprises an ApoE genotype selected from among ApoE ε3/ε3, ApoE ε2/ε4, ApoE ε3/ε4 and ApoE ε4/ε4.

11. The method of claim 10, wherein the rhApoE2 protein is administered intravenously.

12. The method of claim 10, wherein the metabolic disease is selected from the group consisting of diabetes, obesity, cardiovascular disease, and Alzheimer's disease.


SEQUENCE LISTING (3 sequences)

SEQ ID NO: 1 - protein, 299 aa, synthetic construct
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 7 aa, synthetic construct
  SITE 1: note = Acetylated residue
  SITE 7: note = Amidated residue
CDTPPVC

SEQ ID NO: 3 - protein, 6 aa, synthetic construct
SHAVSS